SEQUENCE LISTING (1 sequence)

SEQ ID NO: 1 - DNA, 14 bp, Artificial Sequence
  oligonucleotide
acaaaaaaaaaaaa

(19) United States
(12) Patent Application Publication
(10) Pub. No.: US 2014/0065223 A1
(43) Pub. Date: Mar. 6, 2014

(54) PERFLUORINATED COMPOUNDS FOR THE NON-VIRAL TRANSFER OF NUCLEIC ACIDS

(71) Applicant: Konstanze Schäfer, Berlin (DE)

(72) Inventor: Konstanze Schäfer, Berlin (DE)

(73) Assignees: Jeanette Libera-Koerner, Berlin (DE); Konstanze Schaefer, Berlin (DE)

(21) Appl. No.: 14/008,950

(22) PCT Filed: Mar. 29, 2012
(86) PCT No.: PCT/EP12/55639

§ 371 Date: Nov. 7, 2013

(30) Foreign Application Priority Data
Mar. 31, 2011 (DE) 10 2011 016 334.4
May 9, 2011 (DE) 10 2011 101 361.3
Aug. 26, 2011 (DE) 10 2011 112 191.2
Oct. 20, 2011 (DE) 10 2011 117 390.4

(51) Int. Cl. C12N 15/85

(52) U.S. Cl. CPC C12N 15/85
USPC 424/489; 536/23.1; 514/44 R

(57) ABSTRACT

The invention relates to a compound of general formula (I): A-B-C(F, G′)-D-E-F-G-A′ or a structure of general formula (II): A-B-C-(F′, G′)-D-B-E-F-G-A′ (II), wherein -A is at least one molecule selected from the group of the perfluorocarbons (PFCs), perfluorinated silicon compounds, and/or further perfluorinated compounds, -B is at least one predetermined breaking point in the form of a physically, chemically, or enzymatically severable bond, -C is absent or at least one linker molecule, -D is absent or at least one spacer molecule, -E is at least one molecule selected from the group containing nucleobases, nucleosides, nucleotides, oligonucleotides, nucleic, acids, modified nucleobases, modified nucleosides, modified nucleotides, modified oligonucleotides, modified nucleic acids, monomers of peptide nucleic acids, oligomers or peptide nucleic acids and peptide nucleic acids or other nucleic acid analogs, -F, F′ is absent or at least one ligand, -G, G′ is absent or at least one marker molecule, -A′ is absent or has the meaning of A, and wherein the compounds i), ii), iii), iv), v), vi) are excluded. The invention farther relates to the use of said compound for the non-viral transfer of molecule E into a cell, to a pharmaceutical composition containing said compound, and to the use of said pharmaceutical composition.

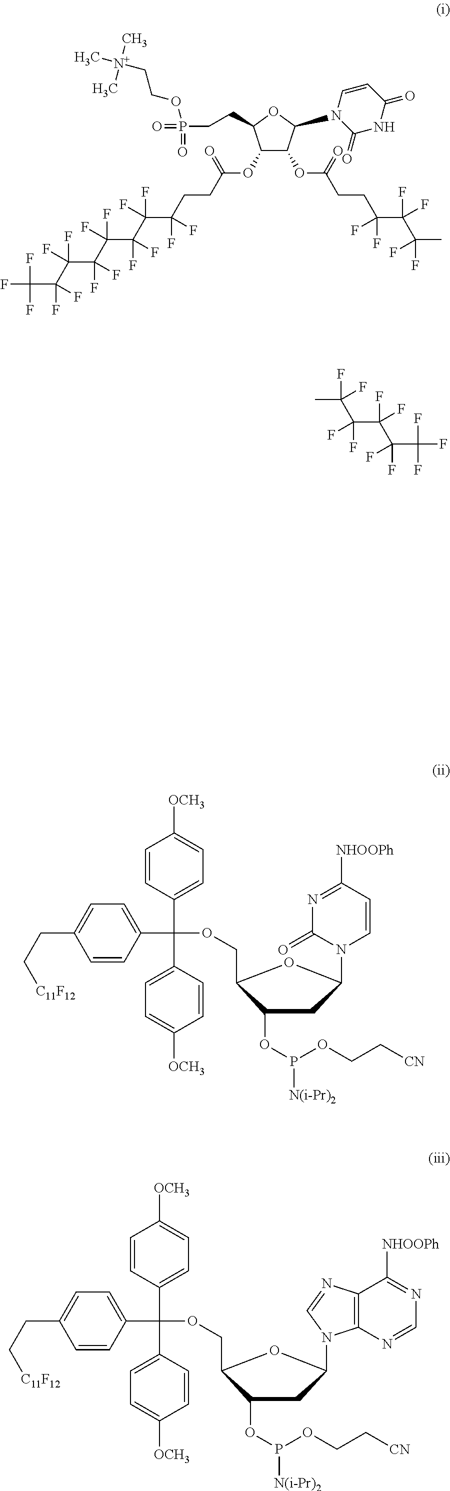

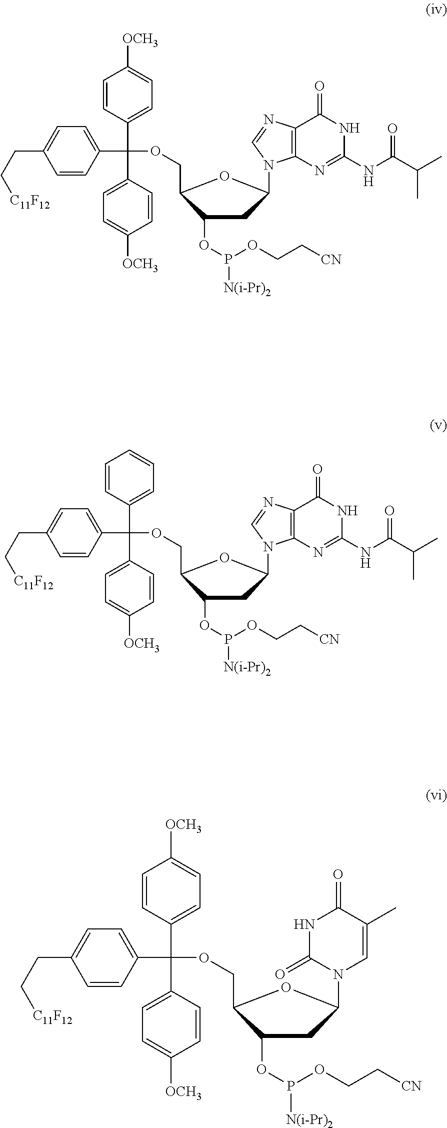

The subject matter of the present invention is a compound according to claim 1, the use thereof according to claim 15, a pharmaceutical composition according to claim 16 and the use thereof according to claim 18.

Non-viral gene transfer is an important area of focus in basic research and in medicine. Possible applications arise particularly in relation to classic hereditary diseases and acquired genetic diseases (e.g., HIV, chronic infectious diseases, tumors, heart and circulatory diseases). In the past, attempts to establish gene therapy in medicine focused above all on viral vectors. However, they are associated with substantial drawbacks. The applications are not sufficiently safe and, what it more, trigger immune responses after one-time application in the body that make a second application impossible. Beyond that, incidents have been reported time and time again in which patients became very ill or even passed away as a result of the treatment.

One alternative to viral gene transfer could be non-viral gene transfer. However, all of the methods known thus far are so inefficient that they are not used in medicine. The non-viral transfer methods include all methods in which no viruses are involved.

The transfer of naked DNA or RNA has already been researched but offers few possible practical applications in the previous form, since transfusion is performed into open tissue or injection is performed into the bloodstream, and RNA and DNA are very fragile in relation to nucleases. What is more, the transfection rates are very low.

In order to work around the abovementioned problems, non-viral nucleic acid transfer by means of DNA or RNA complexes with cationic polymers (e.g., PEI, PEG, PLL, PLA) or with cationic lipids (e.g., CTAB, DOTMA, DOTAP) is gaining in importance. The positive charge of such molecules is used to neutralize the negative charge of the sugar-phosphate structure of the nucleic acid and facilitate absorption through the cell membrane into the cytoplasm of the cell. There are numerous patents on these methods. However, the investigational results in this context only mark the beginning of a trend. After all, besides the still unsatisfactory transfection rates, the toxicity of these polymers and lipids represents a crucial obstacle for the cell. Apart from that, these complexes tend to clump within the cytoplasm, since the biodegradability of the polymer is too low. The loading rates with DNA or RNA increase with the level of the positive charge of the polymer or the lipid. But it is precisely these highly positively charged molecules that have proven to be especially toxic to cells. In order to reduce the toxicity of these cationic polymers and lipids, they are increasingly being combined with hydrophilic polymers, although no outstanding improvement is achieved in this way.

Apart from their low efficiency, previously known transport molecules for non-viral gene transfer have a second drawback in common: They remain in the cytoplasm after transport into the cell and accumulate there or react with cell molecules, or they have negative effects on the cell membrane.

Furthermore, research is being done in modifying nucleic acid building blocks so as to make them suitable for the non-viral transfer of nucleic acids. For instance, WO 2008/039254 and patent US 2010/0016409 describe RNA particles that are double-stranded in part or in whole or are present in other specific conformations and are optionally linked to other molecules. These RNA particles, which have greater stability than single-stranded mRNA due to their conformation, are proposed for non-viral gene transfer. The advantage of these molecules is that they are very small and can also pass through very fine capillary blood vessels. What is more, there is consequently hardly any danger of the clumping that often occurs with relatively large polymer complexes. One drawback of this molecule is that, besides the therapeutic sequences, additional sequences have to be built into the mRNA molecule that are intended to lead to the self-aggregation of certain areas, thus resulting in such RNA conformations as hair needle, nano-ring, quadratic and other structures occur. Although the RNA molecules have a longer life span within the organism than purely single-strand mRNA, the availability of these double-stranded conformations for translation to the ribosomes has not yet been demonstrated.

EP 1 800 697 B1 describes a modified mRNA whose G/C content is higher compared to the wild type and that at least one codon of the wild-type sequence that codes for a tRNA that is relatively rare in the cell is exchanged for a codon that codes for a tRNA that is relatively common in the cell. The mRNA modified in this way is additional altered such that at least one nucleotide analog from the group consisting of phosphorthioates, phosphoramidates, peptide nucleotides, methyl phosphonates, 7-deazaguanosine, 5-methyl cytosine and inosine is incorporated which have already been used in several other RNA methods (siRNA). The method is described for sequence-altered mRNAs from original wild-type peptides.

Document WO 99/14346 also describes an mRNA stabilized through sequence modifications, particularly the reduction of the C- and/or U-content through base elimination or base substitution.

The U.S. Pat. No. 5,580,859 and U.S. Pat. No. 6,214,804 describe transient gene therapy constructs that require an expression vector and emerge from DNA constructs.

WO 02/098443 describes mRNAs that code for a biologically active peptide that is either not formed in the patient to be treated or only to an insufficient, faulty extent and hence does not trigger an immune response.

Another development in the area of non-viral gene therapy is “microbubble” technology, in which stabilized protein microspheres filled with nucleic acid (Kausik Sarkara et al., J. Acoust. Soc. Am. 118, Jul. 1, 2005, pages: 539-550) or sugar microspheres (Schlief et al., Ultrasound in medicine & Biology, Volume 22, Issue 4, 1996, pages 453-462) are additionally filled with ultrasound gases. It had been observed that ultrasound contrast media lead to an intensification of cavitation as a result of which the cell membrane is transiently permeabilized (Tachibana et al., Echocardiography. 2001 May; 18(4):323-8. Review). This lead to an increased absorption of the non-viral gene transfer constructs into the cell. Nonetheless, the efficiency of viral gene transfer was not achieved.

The ultrasound method with contrast media is also being increasingly used in order to increase the efficiency of viral gene transfer (Blomley, 09/2003, Radiology, 229, 297-298).

In addition to a series of other gases, perfluorocarbon gases have proven to be especially suitable for “microbubble” technology. As a result of their highly lyophilic properties and their extremely low surface tension, they are highly suited to enabling the integrity of the cell membrane to be disturbed and substances to thus pass through. These are pure perfluorocarbons that are not bound in some way woth other components. Experiments on non-viral gene transfer by means of pure perfluorocarbons have shown, however, that the nucleic acids from pure perfluorocarbons diffuse away before entering the cell. In this way, nucleic acids can only be taken along as a result of random events.

All of the previous solutions are still far from the efficiency of viral gene transfer. There is great interest in the development of a non-viral transfer system for nucleic acids into the cell the effectiveness of which, for one, is at least equal to the effectiveness of viral gene transfer, and the components of which, for another, do not accumulate in the cell, do not react with the cell molecules and do not have a harmful effect on the cell membrane.

Accordingly, an object of the present invention is to provide a stable compound that overcomes the drawbacks of viral gene transfer and is suited to non-viral gene transfer.

This object was achieved accordingly through the provision of a compound for the non-viral transfer of nucleotide building blocks with the features of claim 1.

The compound according to the invention comprises a structure of general formula (I)

A-B-C(F′,G′)-D-E-F-G-A′ (I)

or a structure of general formula (II)

A-B-C(F′,G′)-D-B-E-F-G-A′ (II)

wherein
-A is at least one molecule selected from the group of the perfluorocarbons (PEC), perfluorosilicon compounds and/or other perfluorinated compounds,
-B is at least one predetermined breaking point in the form of a physically, chemically or enzymatically several bond,
-C is absent or at least one linker molecule,
-D is absent or at least one spacer molecule,
-E is at least one molecule selected from the group containing nucleobases, nucleosides, nucleotides, oligonucleotides, nucleic acids, modified nucleobases, modified nucleosides, modified nucleotides, modified oligonucleotides, modified nucleic acids, peptide nucleic-acid monomers, peptide nucleic acid oligomers and peptide nucleic acids or other nucleic acid analogs,
-F, F′ is absent or at least one ligand or a recognition sequence,
-G, G′ is absent or at least one marker molecule,
-A′ is absent or has the meaning of A, and wherein the compounds

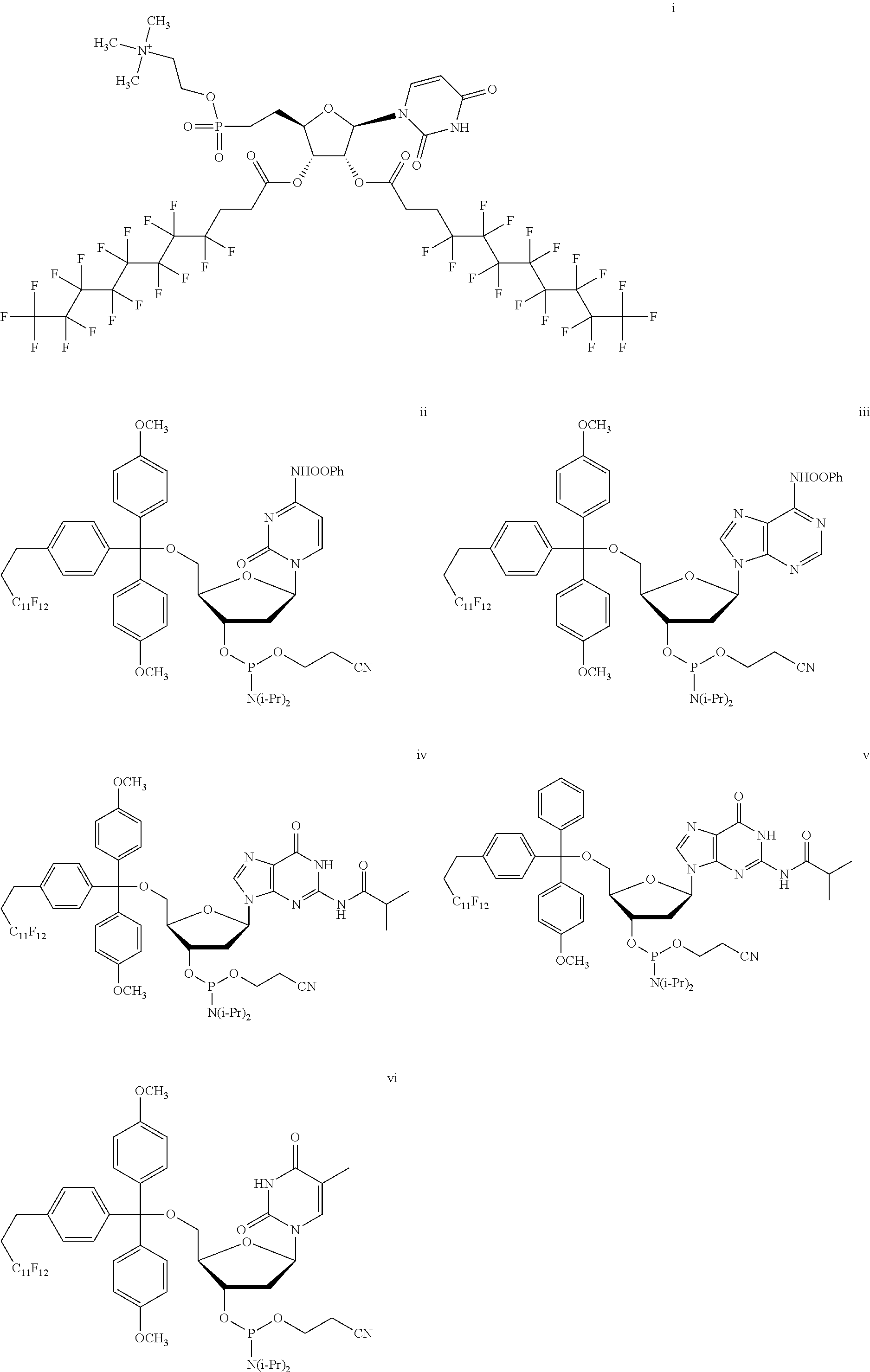

are excluded.

The molecules A, B, C, D, E, F, F′, G, G′ and A′ are preferably each linked together via covalent bonds. However, it is conceivable for the individual molecules or molecule groups of the compound according to the invention to be linked together in whole or in part by ionic bonds.

Accordingly, a new, promising substance group for non-viral gene transfer are molecule compounds particularly composed of perfluorocarbons (PFCs) and, for example, nucleic acid building blocks that are linked together via a predetermined breaking point, so-called perfluorinated nucleic acid building blocks.

The advantages of perfluorinated nucleic acid building blocks are as follows:

1) As a result of their highly lyophilic effect under physiological conditions (more lyophilic than fatty acids) and even more due to their extremely low surface tension, these molecules attach quickly to the surface of the cell membrane. There, they are absorbed into the cell through regularly occurring processes such as pinocytosis, phagocytosis, endocytosis or endocytosis-independent paths. Here, the PFCs are the transport system into the cell for the otherwise not readily absorbable nucleic acid building blocks.

2) Due to their strong C—F bond, PFCs are very inert and do not react with cell molecules.

3) After breaking off from the nucleic acid building blocks, PFCs are small, uncharged lyophilic molecules which, depending on the concentration gradient, can exit the cell passively.

4) Through the medical application of PFCs as oxygen carriers/blood substitutes in humans, it has been shown that PFCs are excreted from the body through the lungs, kidneys and skin.

5) Given that they have already been approved as blood substitutes and as contrast media, the medical approval of PFCs for non-viral gene transfer appears easier.

6) Perfluorinated nucleic acid building blocks exhibit significantly greater absorption into the cell than other substances of non-viral gene transfer and are a true alternative to viral gene transfer.

7) Unlike in viral gene transfer, perfluorinated nucleic acid building blocks do not generate any immune response of the body and can be used as often as desired.

8) In connection with an mRNA transfer, dosing can be achieved due to the limited life span of the mRNA and the unlimited repeatability of the transfer.

Pure perfluorocarbons are originally known from the high-performance lubricants industry. In the pharmaceuticals sector, they have previously been used as blood substitutes above all due to their high degree of oxygen solubility, or even as contrast media. It has also been shown that, in ultrasound applications with non-viral gene transfer microbubbles that were filled with gaseous pure perfluorocarbons, the efficiency of the gene transfer was increased compared to other gases.

However, pure perfluorocarbons are hardly suitable for the non-viral gene transfer of nucleic acids into the cell, since nucleic acids do not adhere to them and can therefore only be taken along by random events. A true bond is needed between the perfluorocarbons and nucleic acid building blocks which can be split by a predetermined breaking point.

Accordingly, the compound according to the invention is characterized by the absorption of the perfluorinated nucleic acid building blocks into the cell, the breaking of the predetermined breaking point between nucleic acid building blocks and perfluorocarbon molecules, the release of cleavage products (nucleic acid building blocks on the one hand and perfluorocarbon molecules on the other) into the cytoplasm and the subsequent diffusion or the active discharging of the perfluorocarbon molecules from the cell, An endocytosis-independent absorption of the perfluorinated nucleic acid building blocks is also possible.

As explained above, perfluorinated nucleic acids are both charged and very lyophilic molecules. The secondary and tertiary structure of these molecules is very well suited to destabilizing the cell membrane and being absorbed into the cell. Under physiological conditions, perfluorocarbons are even more lyophilic than fatty acids and have an extremely low surface tension, which enables the molecule to extend over a large surface of the cell membrane.

By means of a predetermined breaking point between the nucleic acid building block and the perfluorinated portion of the molecule after entering the cell, the perfluorinated portion of the nucleic acid is split off. This usually occurs through acid-labile predetermined breaking points. The increased reduction potential in the cytoplasm and, more so, the low pH value in the endosomes (down to pH=4.5) create the conditions for the hydrolysis thereof.

The predetermined breaking points for this system are sought out such that the cleavage products experience no or few molecular alterations. “Traceless” predetermined breaking points that leave behind an unchanged nucleic acid and a perfluorocarbon group that has obtained its extremely lypophilic and non-polar nature are very suitable for this. One example of such a predetermined breaking point is shown in the following diagram 1:

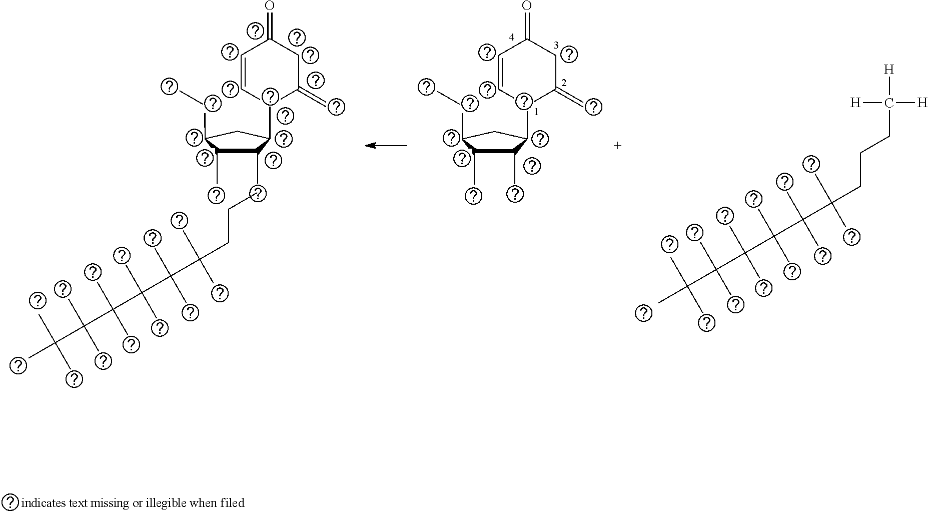

### Diagram 1: Example of a “Traceless” Predetermined Breaking Point

The nucleic acids released into the cytoplasm are freely accessible for the cell. They can have their site of action in the cytoplasm such as, for example, mRNA, siRNA, microRNA, aptamers, antisense RNA and others, or they can be transported into the nucleus, such as DNA with or without nucleus localization sequence, antisense oligonucleotides or individual nucleotides and nucleosides.

The perfluorocarbon molecules with corresponding predetermined breaking point also released into the cytoplasm are uncharged, lyophilic and very small. These characteristics are the conditions for free diffusion along the concentration gradient through the cell membrane. Exocytosis or another release path out of the cell is also possible. Perfluorocarbon molecules are extremely inert and do not react with cell molecules. As long as their molecular structure remains relatively unchanged, they also do not attach to lipids. It is known from the medical use of perfluorocarbons as blood substitutes that they are excreted from the body via the lung and kidney function as well as through the skin.

The perfluorinated nucleic acid building blocks can also be linked with fluorescent dyes in order to follow their path in the cell. By linking with specific ligands or other recognition sequences, the system can be set up for the treatment of special cell types. In principle, the transfer system comprising perfluorinated nucleic acid building blocks can be used for any application in which nucleic acids or modified nucleic acid analogs are to be transported into a cell.

In one embodiment of the present compound, the at least one molecule A is selected from the group of the perfluorocarbons (PFCs) containing straight or branched acyclic or cyclic, polycyclic or heterocyclic aliphatic alkanes, alkenes, alkines, aromatic compounds or combinations of these compounds in which all of the H-atoms are substituted by F-atoms which can optionally also contain at least one non-fluorinated or partially fluorinated substituent in the form of one or more functional groups, aliphatic chains or heteroatoms, particularly Br, I, Cl, H, Si, N, O, S, P or these in conjunction with one or more additional functional groups.

It is also preferred that A be selected from the group of the perfluorocarbons (PFCs) containing C₁-C₂₀, preferably C₁-C₁₀, particularly preferably C₁-C₅₀, very preferably C₁-C₃₀, most preferably C₁-C₂₀ alkanes, alkenes or alkines which can be linear, branched, cyclic, polycyclic or heterocyclic, C₆-C₅₀, preferably C_{c} C₃₀, particularly preferably C₁-C₂₀ aromatic or heteroaromatic systems.

Typically, in relation to the present invention, A-molecules and A-molecule groups can be selected from the PFC group containing —(CnₙF₍₂ₙ₊₂₎₋₁) where n≧1, preferably n=1-20, for example —CF₃, —C₂F₅, —C₃F₇, —C₄F₉, —C₄F₁₁, etc., —(Cnₙ F₂ₙ₋₁) where n÷2, preferably n=2-20, for example —C₂F₃, —C₃F₅, —C₄F₇, etc., —(CnₙF₍₂ₙ₋₂₎₋₁₎ where n≧2, preferably n=2-20, for example —C₂F, —C₃F₃, —C₄F₅, —C₅F₇, etc.

To manufacture the compound according to the invention, it is sensible to use perfluorinated compounds that have suitable functionality in order to enter into a covalent bond with the other molecules, such as B and E. This functionality of the perfluorinated compound used as the starting substance enables, in particular, addition, substitution, esterification, etherification, condensation, etc. Such ligation reactions are known to the person skilled in the art. Preferred functionalities are selected from the group containing halogen alkanes, hydroxyl, ether, amino, sulhydryl, aldehyde, keto, carboxyl, ester and acid amide groups, groups with radicals or ions and molecules from the substance groups of the carboxilic acids, peroxycarboxylic acids, thiocarboxylic acids, sulfonic acids, sulfine acids, sulfene acids, sulfoxides, carboxylic acid salts, sulfonic acid salts, sulfine acid salts, sulfene acid salts, carboxylic acid anhydrides, carboxylic acid esters, sulfonic acid esters, carboxylic acid halogenides, sulfonic acid halogenides, carboxylic acid amides, sulfonic acid amides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halides, nitro compounds, nitroso compounds, azo compounds, diazo compounds, diazonium salts, isocyanates, cyanates, ethers, acid amides and thioethers or compounds that can be reactive due to their multiple bond. Especially preferred are heteroatoms such as Br, I, Cl, H, Si, N, O, S, P, hydroxyl, amino, carboxyl groups, halogen alkanes, carboxylic acid amines, alcohols, hydrazines, isocyanates, thiocyanates and acid amides.

Preferably, as a starting compound for the preparation of molecule A in the compound according to the invention, a substance is used that is suitable for nucleophilic substitution. The starting substance for the preparation of molecule A has a nucleophilic leaving group that is readily recognizable for a person skilled in the art. Accordingly, additional preferred A-molecules can be based on any one of the following starting substances:
-F)CF₂)ₙX, where n=1-50, preferably n=1-10, and X=Br, I, Cl, H or X=Si, N, O, S, P in conjunction with a functional group, particularly C₈F₁₇1, C₈F₂₇Br;
-F(CF₂)ₙ)—CH₂)ₙX, where n=1-50, preferably n=1-10, m=1-26, preferably m=1-6 and X=Si, N, O, S, P, Br, I, H;
-F(CF₂)ₙ—O_{b}—CH═CH₂, where n=1-50, preferably n=1-10, and b=0 or 1, preferably b=0;

-C₆F₁₃CH₂CH₂Mgl, (C₆F₁₃CH₂CH₂)₃SnPh, (C₆F₁₃CH₂CH₂)₃SnBr, (C₆F₁₃CH₂CH₂)₃SnH;
-C₂F₅I, C₃F₇Br, C₄F₉1, C₅F₁₁Br, C₆F₁₃Br, C₈F₁₅Br, C₁₀F₂₇I,
-C₄F₉CH═CHC₄F₉, C₈F₁₆C₁₂, C₁₀F₁₉N, C₆F₁₉Br, C₉F₂₁N, C₁₀F₂₁Br, C₁₁F₂₂N₂O₂, C₆F₁₃CH═CHC₆F₂₃, C₁₂F₂₇N, C₁₂F₂₇N, C₁₆F₂₅Br;
-C₈F₁₇1, C₈F₂₇Br.

Additional preferred starting substances of the A-molecule from the group of the perfluorohydrocarbons used in relation to the present invention are:
-perfluorinated cholesteryl and adamantyl compounds, perfluorinated cis-eicosenoic, perfluorinated aromatic compounds, perfluorinated pyrenes, perfluorinated glycerides; and

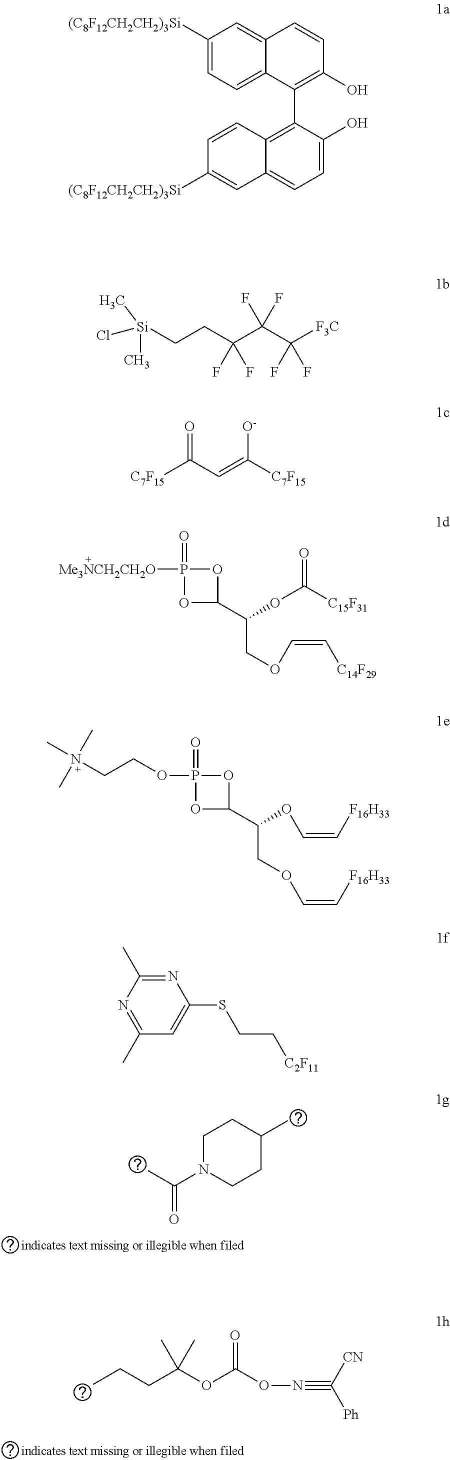

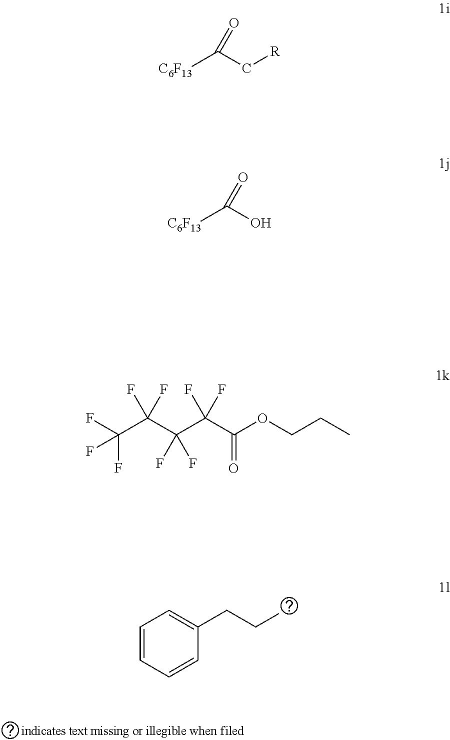

-for linking in the form of an ionic bond, the following PFCs can be used, for example:

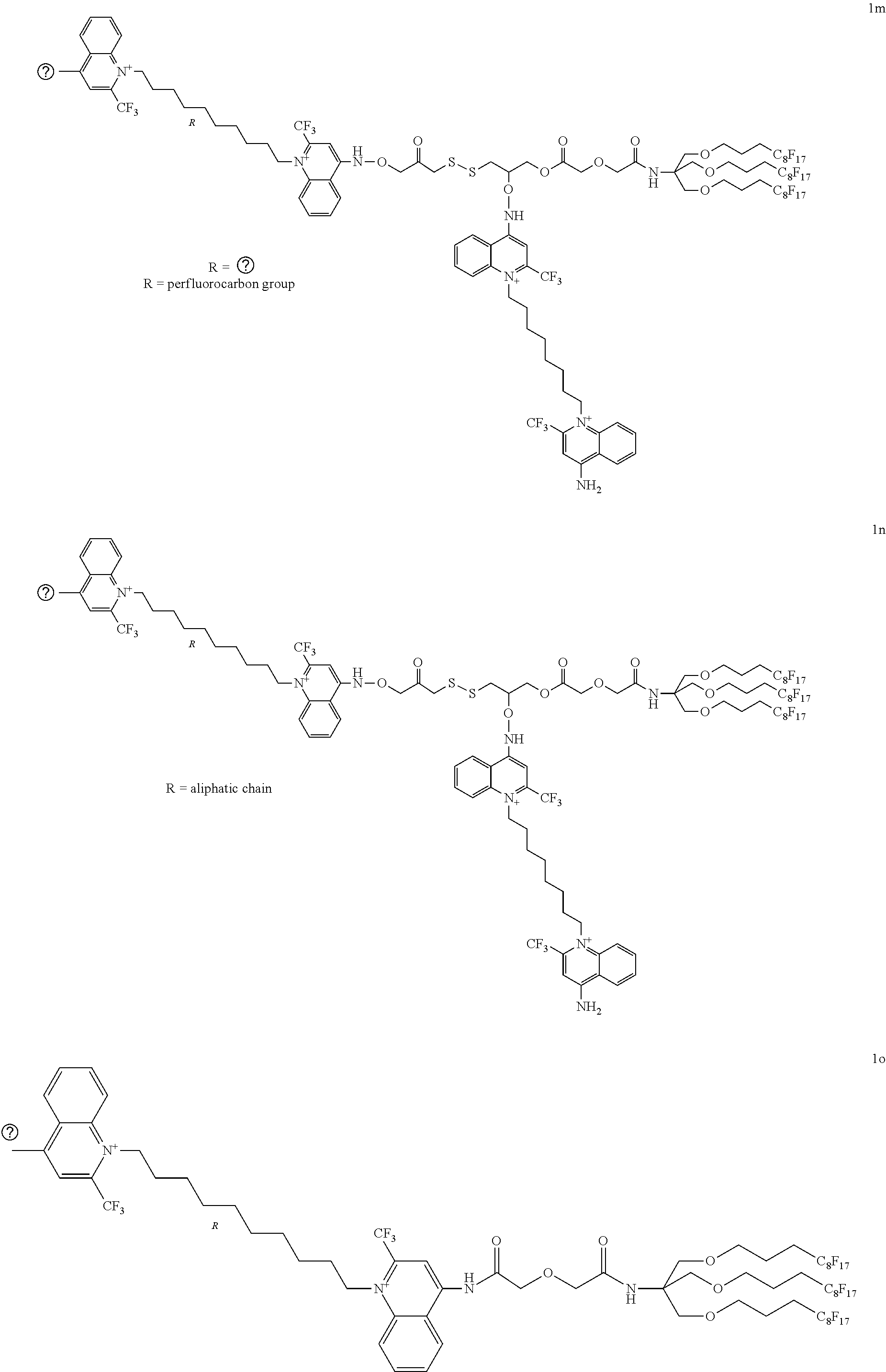

where R=perfluorocarbon group or aliphatic chain;

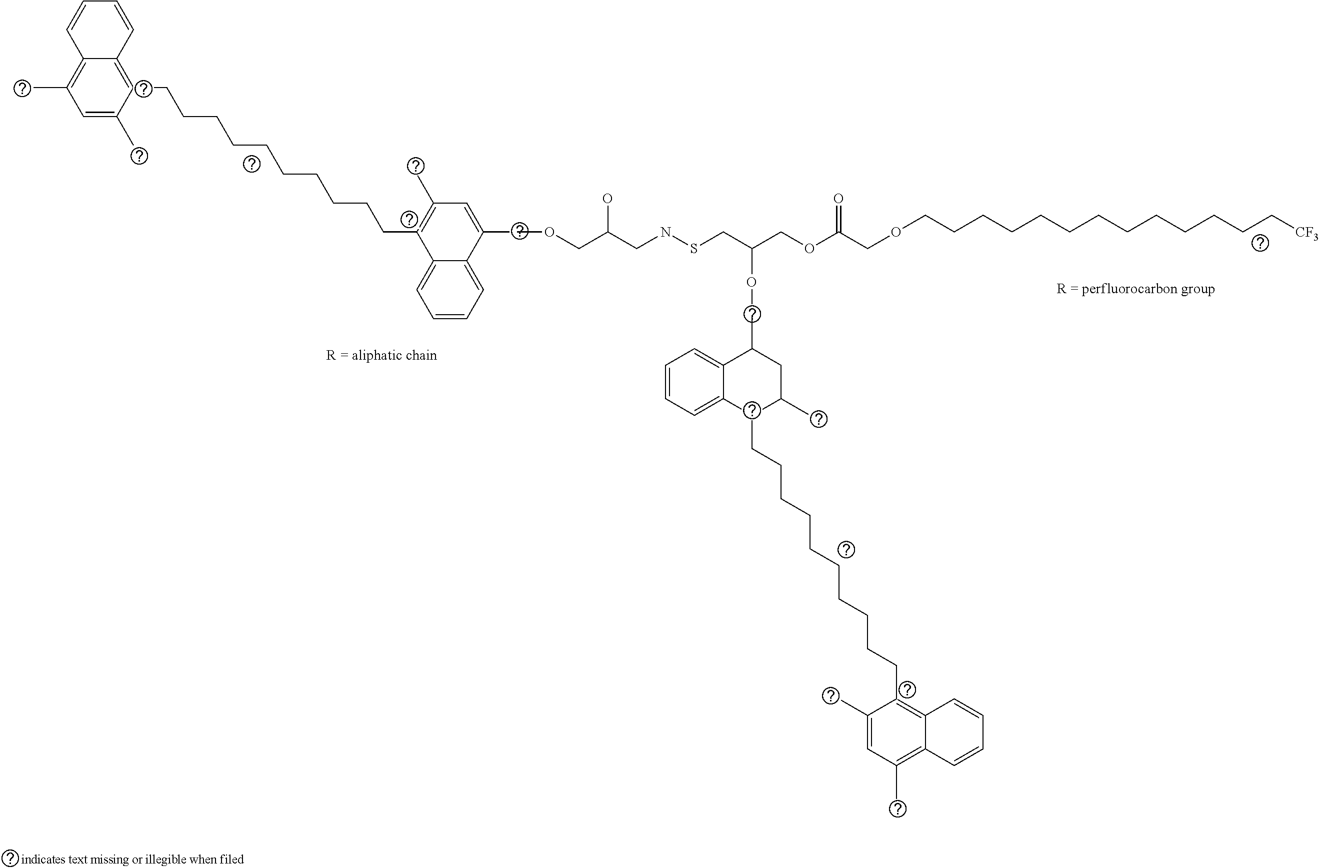 [SMILES: Cc1cc(C)c2ccccc2c1CCCCCCCCCCc1c(C)cc(COCC(O)CNSCC(COC(=O)COCCCCCCCCCCCCCC(F)(F)F)OCC2CC(C)C(CCCCCCCCCCc3c(C)cc(C)c4ccccc34)c3ccccc32)c2ccccc12]

Another embodiment of the present invention is that the at least one molecule A is selected from the group of the perfluorosilicon compounds containing straight or branched acyclic or cyclic, polycyclic or heterocyclic aliphatic silanes in which all H-atoms are substituted by F-atoms, which optionally additionally contain non-fluorinated or partially fluorinated substituents with one or more functional groups or heteroatoms, particularly Br, I, Cl, H, Al, N, O, S, P, or these in combination with one or more additional functional groups.

Preferably, perfluorosilicon compounds from the group containing Si1-Si200, preferable Sit-Si100, particularly preferably Si1-Si50, very preferably Si1-Si30, most preferably Si1-Si20 perfluorinated silicon compounds are used. The perfluorosilicon compounds are also functionalized with suitable substituents as in the case of the PFCs.

Additional preferred functionalized perfluorosilicon compounds can be selected from the following group:
-siloxanes of the general formula —[—SiR₂—O-]ₙ, where n≧1 and R=perfluorinated carbons or F,
-siloxanoles of the general formula HO-A-[—SiR₃R₄—O]—SiR₃R₄R₅, where R_{3,4,5}=F or —F(CF₂)ₙ, where n=1-10 and A=alkyl chain
-perfluorinated silicates, silicic acids, sodium silicates, polysilazanes, silicides, silicon tetrahalogenides, silicons, silicon oils, zeolites, zirconium silicates,
-silanes of the general formula C—CH₂—Si(OR)₃, X being a leaving group suitable for nucleophilic substitution, where X═Cl, Br, I, H, OH or functional groups comprising amino, vinyl, carbamato, glycidoxy, methylalkoxy, phenyl or acetoxy groups and R═F or —F(CF₂)ₙ, where n=1-10,
-silanes of the general formula X—Si (CF₃)₃ or X—Si(R)₃, X being a leaving group suitable for nucleophilic substitution, where X═Cl, Br, I, H, OH or functional groups such as amino, vinyl, carbamato, glycidoxy, methylalkoxy, phenyl or acetoxy groups and R═F or —F(CF₂)ₙ, where n=1-10.

In yet another embodiment, the at least one molecule A is selected from¹ the group of other perfluorinated compounds, the molecule A being based on compounds that are selected from among NF₃, N₂F₄, SNF₃, CF₃SN, SF₄, SF₆, perfluorinated nitrogen-sulfur compounds. ¹Translator's note: This phrase also contains the verb “contains” [“enthält”] but appears to be have been left unintentionally.

It is also preferred that the present compound contain two or more molecules A selected from the group of the perfluorocarbons (PFC), perfluorosilicon compounds and other perfluorinated compounds.

Typically, the above-described functionalized perfluorinated compounds can be integrated into several units. Reference is made to the following molecules as examples:

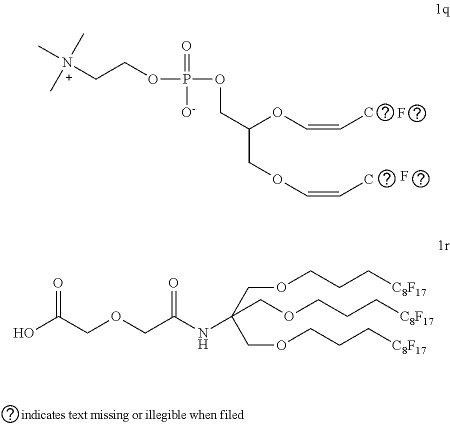

The linking of these molecules to the other molecules included in the compound according to the invention can be done via NH₂ groups or OH groups or other suitable groups.

In a variant of the present compound, the at least one predetermined breaking point B is embodied in the form of an acid-labile group, particularly in the form of a glycosidic bond, at least one disulfide bridge, at least one ester group, ether group, peptide bond, imine bond, hydrazone bond, acylhydrazone bond, ketal bond, acetal bond, cis-aconitrile bond, trityl bond, beta-D-glucosylceramide, and/or dithiothreitol.

Predetermined breaking points have two important functions within the constructs of PFCs and nucleic acid building blocks. For one, the predetermined breaking points serve the purpose of so-called “leakage.” Here, the particles of the PFC-nucleic acid compounds attach to the endosome membrane of cells and must then be released from these endosomes. This occurs through the destruction of the integrity of the membrane, which is brought about by the splitting of the constructs. For another, the group of the PFC molecule has to be split off in order to make the nucleic acid building blocks available to the cell. Here, the acid-labile predetermined breaking points such as the gylycosidic bonds, disulfide bridges, esters or ethers listed above are hydrolyzed (split) under acidic conditions chemically or by hydrolases/esterases, for example at the 2′ position of the nucleotide or at other locations.

More complex predetermined breaking points can be present in the form of specific substrates for enzymes, pH- and photosensitive lipid functions, molecules with release by ultrasound action or temperature-controlled lipid modifications, as well as other bonds that can be split under physiological conditions.

Reference is made to the following predetermined breaking points for the sake of example:
-Plasmalogen perfluoride, cleavable by light

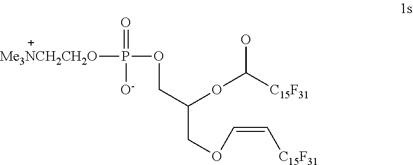

-pH-sensitive perfluorinated vinyl ether functions

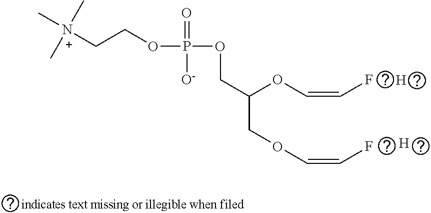

-Orthoesters cleavable by rearrangement

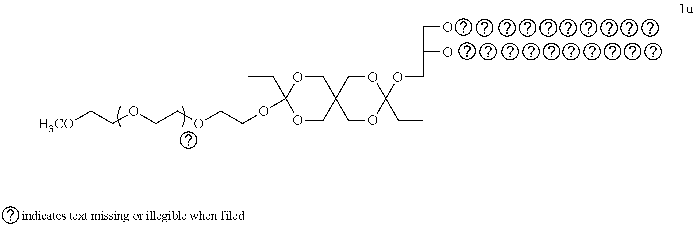

In one embodiment of the present invention, the at least one linker molecule C is selected from the group containing straight or branched acyclic or cyclic, polycyclic or heterocyclic aliphatic alkanes, alkenes, alkines, aromatic compounds or combinations of these compounds with functional groups.

The linker molecule C used is preferably used in order to make available in the present compound one or more bond sites for other, additional molecules such as, for example, marker molecules G′ and/or ligands F′ or other recognition sequences.

Preferably, the linker molecule C is selected from a group containing, particularly, halogen alkanes, hydroxyl, ether, amino, sulhydryl, aldehyde, keto, carboxyl, ester and acid amide groups, groups with radicals or ions and molecules from the substance groups of the carboxylic acids, peroxycarboxylic acids, thiocarboxylic acids, sulfonic acids, sulfinic acids, sulfenic acids, sulfoxides, carboxylic acid salts, sulfonic acid salts, sulfinic acid salts, sulfenic acid salts, carboxylic acid anhydrides, carboxylic acid esters, sulfonic acid esters, carboxylic acid halogenides, sulfonic acid halogenides, carboxylic acid amides, sulfonic acid amides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halogenides, nitro compounds, nitroso compounds, azo compounds, diazo compounds, diazonium salts, isocyanates, cyanates, isocyanates, thiocyanates, isothiocyanates, hydroperoxides, peroxides or compounds that can be reactive due to their multiple bond, or functionalized perfluorohydrocarbons with iodine, bromine or sulfur atoms, carbamates, thioether or disulfide groups, glycerol, succinyl glycerol, phosphate groups as well as functionalized perfluorohydrocarbons with other groups or atoms via which a link can be established to nucleosides, nucleotides, oligonucleotides, nucleic acids, modified nucleosides, modified nucleotides, modified oligonucleotides, modified nucleic acids, peptide nucleosides, peptide nucleotides, peptide oligonucleotides, peptide nucleic acids or pharmaceutical substa nces.

Preferred linker molecules C are based on hydroxyl or amino groups, carboxyl groups, esters, ethers, thioethers, thioesters, carboxylic acid amides, compounds with multiple bonds, carbamates, disulfide bridges and hydrazides, halogen alkanes, sulhydryl, aldehyde, keto, carboxyl, ester and acid amide groups, thiols, amines, imines, hydrazines, or disulfide groups, glycerol, succinyl glycerol, orthoesters, phosphoric acid diesters and vinyl ethers, ester and ether groups and disulfide bridges being very especially preferred.

As described above, the linker molecule C can be used to link other markers G′ and/or ligands F′ or other recognition sequences which substantially comprise the same groups as the ligand F and marker G defined below but differ from these nonetheless by the arrangement within the present compound.

Suitable examples here of the marker G′ are fluorescent dyes such as Dil, DilC, DiO, fluoresceins, rhodamines, oxacines, fuchsines, pyronines, acridines, auramines, pararosanilines, GFP, RFP, DAPI or peroxidase dyes such as ABTS.

Ligands and recognition sequences are required for specific transfer in certain cell types, since they bind to receptors on the cell surface and thus enable specific entry into the cell. Ligands are generally bound to the construct via accessible side or terminal amino groups. Bonds via other groups are possible, however.

Transferrin, folic acid, galactose, mannose, epidermal growth factor, RGD peptides, biotin, and other substances can be used here as suitable ligands F′. Examples of recognition sequences are nucleus localization sequence or sequences for endocytosis-independent absorption.

In another variant of the present invention, the at least one spacer molecule D is selected from the group containing straight or branched aliphates with one or more functional groups, it also being possible to use the spacer molecule D as linker molecule C.

In the present compound, a spacer molecule is used in order to give the various groups in the molecule more space to prevent steric impediments or in order to weaken the negative charge of the fluorine building blocks to other molecule areas. Especially preferably, the spacer molecule D is selected from the group of the fatty acid alcohols, fatty acid diols and fatty acid polyols.

In a preferred embodiment of the present invention, nucleobases are used as molecule E that are selected from the group containing adenine, guanine, hypoxanthine, xanthine, cytosine, uracil, thymine, modified nucleobases such as 5-bromouracil, 5-fluorouracil, zidovudines, azidothymidines, stavudine, zalcitabine, diadenosine, idoxuridine, fluridine and ribavirin, azidothymidine, zidovudine, 5-methyluracil, 5-methylcytosine, 5-fluorocytosine, 5-bromocytosine, 2-aminopurine and “spiegelmers” thereof, the nucleobases adenine, guanine, cytosine, uracil and thymine being especially preferred.

If nucleosides are used as molecule E, as is preferred, then the nucleosides are selected from the group containing adenosine, guanosine, cytidine, 5-methyluridine, uridine, deoxyadenosine, deoxyguanosine, thymidine, deoxyuridine, deoxycytidine or modified nucleosides such as 2-thiocytidine, N4-acetylcytidine, 2′-O-methylcytidine, 3-methylcytidine, 5-methylcytidine, 2-thiouridine, 4-thiouridine, pseudouridine, dihydrouridine, 5-(carboxyhydroxymethyl)-uridine, 5-carboxymethylaminomethyl-uridine, 5-methylaminomethyluridine, 5-methoxy-carbonylmethyl-uridine, 5-methoxyuridine, 2′-β-methyluridine, ribothymidine, 1-methyladenosine, 2-methyladenosine, N6-methyladenosine, N6-isopentenyladenosine, 2′-O-methyladenosine, inosine, 1-methylinosine, 1-methylguanosine, N2-2-methylguanosine, N2-2,2-dimethylguanosine, 7+-methylguanosine, 2′-O-methylguanosine, queuosine, β-D-galactosylqueuosine, β-D-mannosyl-queuosine, archaeosine, 2′-O-ribosyladenosinphosphate, N6-threonylcarbamoyladenosine, lysidine, nicotinic acid, riboflavin and pantothenic acid, NADPH, NADH, FAD, coenzyme A, and succinyl coenzyme A, puromycin, aciclovir, ganciclovir and “spiegelmers” thereof, the nukleosides adenosine, guanosine, uridine, cytidine, deoxyadenosine, deoxyguanosine, deoxycytidine, deoxythymidine being especially preferred.

In another preferred embodiment of the present compound, nucleotides are used as molecule E that are selected from the group containing AMP, GMP, m5UMP, UMP, CMP, dAMP, dGMP, dTMP, dUMP, dCMP, cAMP, cGMP, c-di-GMP, cADPR, ADP, GDP, m5UDP, UDP, CDP, dADP, dGDP, dTDP, dUDP, dCTP, ATP, GTP, m5UTP, UTP, CTP, dATP, dGTP, dTTP, dUTP, dCTP or modified nucleotides that originate from the above-described building blocks, nucleotides with modifications on the sugar-phosphate structure, zwitterionic oligonucleotides as well as nucleotides in which the phosphate has been replaced by methyl phosphonate or a dimethyl sulfone group, AMP, GMP, m5UMP, UMP, CMP, dAMP, dGMP, dTMP, dUMP, dCMP being especially preferred.

It is further preferred to use deoxyribonucleic acids, ribonucleic acids and modified nucleic acids as molecule E, such as, for example, nucleoside phosphorothioates, zwitterionic nucleoc acids, nucleic acids in which the phosphate has been exchanged for a methyl phosphonate or dimethyl sulfone group, bridged nucleic acids (locked nucleic acids), “spiegelmers,” nucleic acids in which the ribose-phosphodiester backbone has been exchanged for various polymeric constructs, such as a hexitol-based backbone strand or a nucleic acid analog based on glycerin units), morpholino oligonucleotides, phosphorthioate deoxyribonucleic acid, cyclohexene nucleic acids, N3′-P5′-phosphoramidates, tricyclo-deoxyribonucleic acids, morpholino phosphoramidate nucleic acids, threose nucleic acids, with nucleoside phosphorothioates, phosphorthioate deoxyribonucleic acid being especially preferred.

It is also preferred to use, as molecule E, monomers of the peptide nucleic acids such as (Fmoc)-adenine-(Bhoc)-OH, (Fmoc)-cytosine-(Bhoc)-OH, (Fmoc)-guanine-(Bhoc)-OH, (Fmoc)-thymine-(Bhoc)-OH or peptide nucleic acids in which the complete ribose phosphodiester backbone has been replaced by a peptidic, achiral backbone that is based on N-(2-amino-ethyl)glycine subunits in which the bases are linked to the backbone via a carboxymethylene unit, with (Fmoc)-adenine-(Bhoc)-OH, (Fmoc)-cytosine-(Bhoc)-OH, (Fmoc)-guanine-(Bhoc)-OH, and (Fmoc)-thymine-(Bhoc)-OH being especially preferred.

If single-strand or double-strand oligonucleotides and nucleic acids are used as molecule E, as is preferred, then they can have a length of 2 base pairs of up to greater than 1,000,000 bp, the following length ranges each being preferred: 10 to 50 bp, 15 to 25 bp, 25 to 200 bp, 25 to 100 bp, 200 to 300 bp, 200 to 500 bp, 500 to 1500 bp, 800 to 1300 bp, 1500 to 20,000 bp, 1500 to 5000 bp, 3000 to 8000 bp, 20,000 to 1,000,000 bp or 20,000 to 50,000, oligonucleotides with a length between 10 to 50 bp, 200 to 500 bp and 500 to 1500 bp being very especially preferred.

In a variant of the compound, the molecule E is provided with functional groups selected from the group containing hydrazides, halogen alkanes, hydroxyl, ether, amino, sulhydry-, aldehyde, keto, carboxyl, ester and acid amide groups, groups with radicals or ions and molecules from the substance groups of the carboxylic acids, peroxycarboxylic acids, thiocarboxylic acids, sulfonic acids, sulfinic acids, sulfenic acids, sulfoxides, carboxylic acid salts, sulfonic acid salts, sulfinic acid salts, sulfenic acid salts, carboxylic acid anhydrides, carboxylic acid esters, sulfonic acid esters, carboxylic acid halogenides, sulfonic acid halogenides, carboxylic acid amides, sulfonic acid amides, carboxylic acid hydrazides, nitriles, aldehydes, thioaldehydes, ketones, thioketones, oximes, alcohols, phenols, thiols, amines, imines, hydrazines, ethers, esters, thioethers, thioesters, hydrogen halides, nitro compounds, nitroso compounds, azo compounds, diazo compounds, diazonium salts, isocyanates, cyanates, isocyanides, thiocyanates, isothiocyanates, hydroperoxides, peroxides or groups that can be reactive due to their multiple bond or functionalized perfluorohydrocarbons with iodine, bromine or sulfur atoms, carbamates, thioether or disulfide groups, glycerol, succinyl glycerol, phosphate groups or other functional groups with which a bond can be made to a functionalized perfluorohydrocarbon.

The functional groups are to be understood as additional substituents on the nucleotide. For example, an NH₂ group can be introduced at the 2′H position of the deoxyribose of a nucleoside or nucleotide in order to prepare this site for perfluorination, which was originally not usable for perfluorination. An SH group can also be introduced, for example, at the 2′OH position of the ribose in order to later create a disulfide bridge.

Preferred substituents or functional groups at molecule E are —OH, —NH₂ and —SH groups, hydrazides, halogen alkanes, sulhydryl, aldehyde, keto, carboxyl, ester and acid amide groups, ethers, thioesters, and thioethers.

The ligand F present in the present compound is preferably selected from a group containing transferrin, folic acid, galactose, lactose, mannose, epidermal growth factor, RGD peptides, biotin, and other substances that enable a specific entry of the present compound into the cell.

The marker molecule G present in the present compound is preferably selected from a group containing fluorescent dyes such as Dil, DilC, DiO, fluoresceines, rhodamines, oxacines, fuchsines, pyronines, acridines, auramines, pararosanilines, GFP, RFP, DAPI, peroxidase dyes such as ABTS and other substances that enable the molecule to be tracked during metabolism.

The present compound is particularly suitable and can be used for the non-viral transfer of at least one molecule E into at least one cell of a eukaryotic organism, particularly of animals or humans.

Especially preferably, the present compound is used in the form of a pharmaceutical composition with at least one surface-active substance, suitable surface-active substances being, for example, poloxamers, lecithins or other cell-tolerated surfactants.

The pharmaceutical composition can be present in the form of an emulsion, dispersion, suspension or solution, particularly with an average particle size between 2 nm and 200 μm, preferably between 20 nm and 400 nm, particularly preferably at 50 nm. The particle size can vary depending on the application. Through suitable methods, for example a sonicator, atomization or solvent method, the desired particle size can be set that is favorable for absorption into the cell. The present pharmaceutical composition is also suitable and can be used for the non-viral transfer of at least one molecule E into at least one cell of a eukaryotic organism, particularly of animals or humans.

The present compounds can be manufactured and modified in various ways. In particular, the linking of the molecule E to the perfluorinated compounds such as perfluorohydrocarbons (PFCs) and/or perfluorosilicon compounds can occur at different positions of the molecule E that are accessible for this.

In terms of the present application, a perfluorination side or position is to be understood particularly as the place in the molecule E in which the linking of the molecule E to the perfluorinated molecule A or A′ preferably occurs via a predetermined breaking point B with optional use of a linker molecule C and/or spacer molecule D.

In the following, preferred perfluorination positions are listed for various molecules E: a) perfluorination of nucleobases, nucleosides, nucleotides, b) perfluorination of peptide nucleic acid monomers and oligomers, peptide nucleic acids, c) perfluorination of oligonucleotides.

In a first variant, the perfluorination of nucleobases as molecule E can occur at all accessible places in the molecule, with NH₂ and NH groups being preferred perfluorination sites. The only limitation occurs during the conversion to the nucleoside at the respective NH group (arrow); see diagram 2.

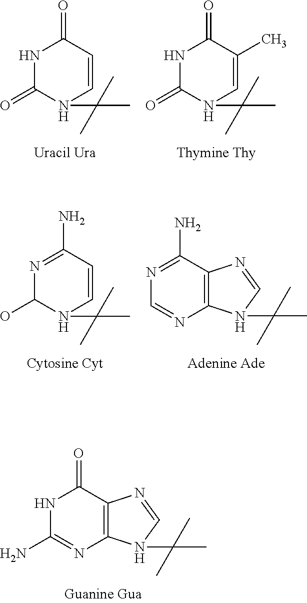

### Diagram 2: Perfluorination Positions in Nucleobases

In a second variant, a perfluorination is also possible at perfluorination sites in the sugar molecule of nucleosides, with preferred perfluorination sites being the 2′, 3′ and/or 5′ position of the ribose. The corresponding positions of the ribose can also be modified in the form of —NH₂ and/or —SH, the preferred perfluorination sites corresponding to 2′-NH₂, 2′-SH, etc.; see diagram 3.

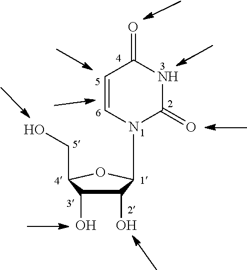

### Diagram 3: Perfluorination Positions in Nucleosides

For the perfluorination of nucleotides, in addition to the perfluorination sites at the ribose in 2′ and 3′ position, according to a third variant there are additional perfluorination sites at the phosphate group, such as at the free OH group or on at least one oxygen atom; see diagram 4.

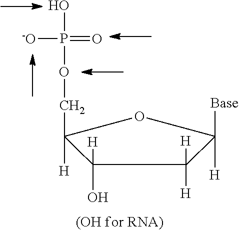

### Diagram 4: Perfluorination Positions in Nucleotides

Alternatively, however, perfluorination sites can also arise on modified sugar-phosphate structures with heteroatoms such as, for example, S or N or others, as shown in diagram 5.

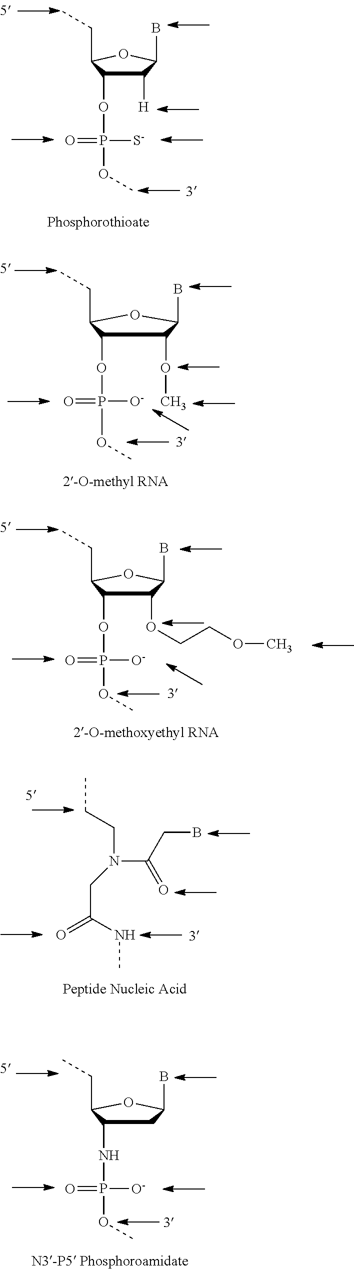

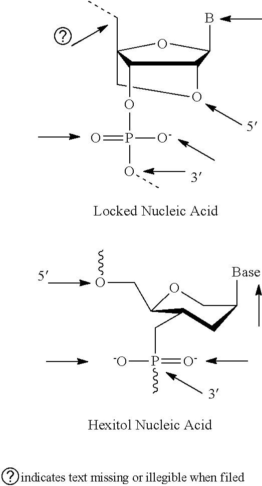

### Diagram 5: Other Perfluorination Positions on Modified Sugar-Phosphate Structures

In a fourth variant, the perfluorination of peptide nucleic acid monomers, peptide nucleic acid oligomers and peptide nucleic acids can occur. These are analogs of nucleic acids. The sugar-phosphate backbone is replaced by a pseudopeptide, for example by aminoethylglycine units that are joined together by neutral amide bonds. They are very stable, since they cannot be broken down by nucleases or by proteases, either. They hybridize more stringently with complementary DNA and RNA sequences as original oligomers, and additional perfluorination sites exist on NH₂ and carboxy groups and on the O-atom, as well as on functional groups of modified peptide structures; see diagram 6.

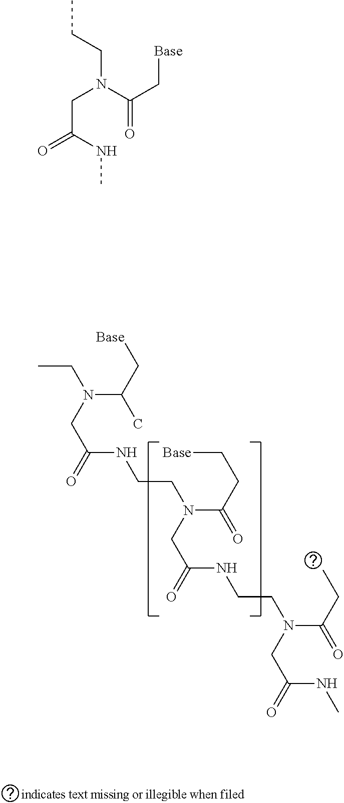

### Diagram 6: Perfluorination Positions on Peptide Nucleic Acid Monomers, Peptide Nucleic Acid Oligomers and Peptide Nucleic Acids

In a fifth variant, the perfluorination of nucleic acid oligomers or oligonucleotides and of nucleic acid macromolecules can occur, with different approaches being possible.

In a first approach, perfluorinated nucleotides are built directly into the desired RNA or DNA sequence, for example by means of solid-phase synthesis or by means of PCR, it being preferred to use perfluorinated nucleotides that are perfluorinated at the 2′ position (i.e., in the 2′ position of the ribose). The reason for this is that, for one, perfluorinations at this site do not lead to chain breakage during polymerization or synthesis and, for another, the interactions between base pairs are not disturbed; see the two upper examples of diagram 7.

While the 2^{x}OH position is simply perfluorinated in RNA molecules, in DNA molecules the 2′H position must first be functionalized, for example by perfluorinatable groups such as NH₂ instead of H or 2′-amino-2′-deoxyuridine.

In a second approach, the incorporation of RNA nucleotides in the DNA sequence or the incorporation of perfluorinated DNA nucleotides at the 5′ or 3′ ends of the oligonucleotide or of the DNA macromolecule; see third example in diagram 7. In this approach, accordingly, perfluorinated compounds such as perfluorinated nucleotides are added to the ends of the finished oligonucleotide preferably by means of chemical synthesis. The nucleotides used can be perfluorinated at any possible position as described above, with 2′- and 5′-perfluorinated nucleotides being preferred. The only exception here is a nucleotide perfluorinated at the 5′ position with C₈F₁₇.

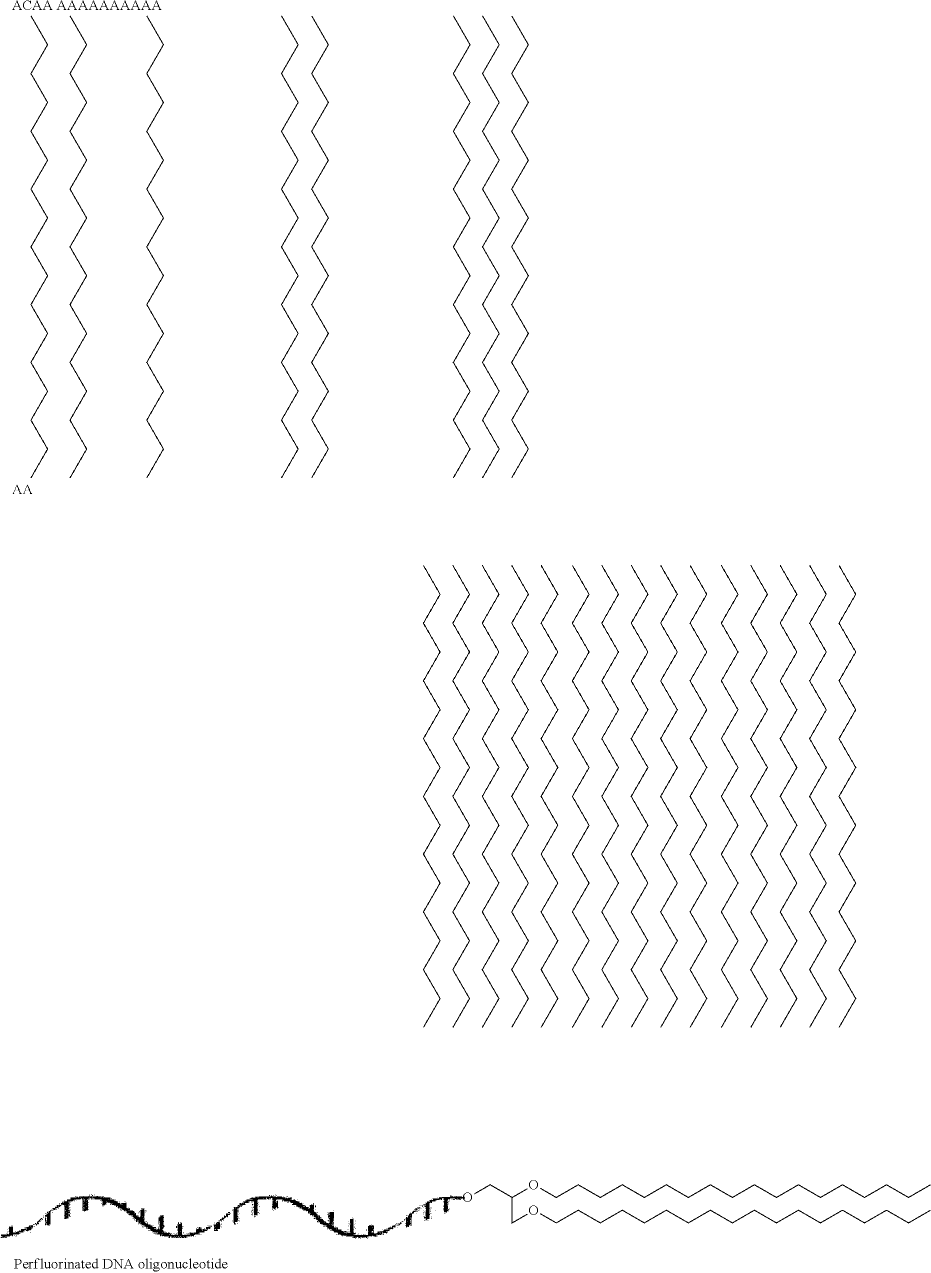

### Diagram 7: Perfluorination Positions of Nucleic Acid Oligomers and of Nucleic Acid Macromolecules

The compounds of the invention can be present in different constructs with different structures, which is to say the compounds can have the following additional basic structures depending on the presence of the molecules C, D, F and G

-A-B-E-F-G, (III)

-A-B-C-B-E-F-G, (IV),

-A-B-C-B-E-F-G, (V),

-A-B-E, (VI),

-A-B-D-B-E, (VII),

-A-B-C-B-E (VIII),

-A-B-E-F, (IX)

-A-B-D-B-E-F, (X),

-A-B-V-B-E-F, (XI),

-A-B-E-G, (XII)

-A-B-D-B-E-G, (XIII),

-A-B-C-B-E-G, (XIV),

-A-B-E-A′- (XV),

-A-B-D-B-E, (XVI),

-A-B-C(F′)-B-E (XVII),

-A-B-C(G′)-B-E, (XVIII)

-A-B-C(F′,G′)-B-E, (XIX),

-A-B-C(F′)-E, (XX),

-A-B-C(G′)-E, (XXI)

-A-B-C(F′,G′)-E, (XXII)

For the sake of example, reference is made to the following compounds:

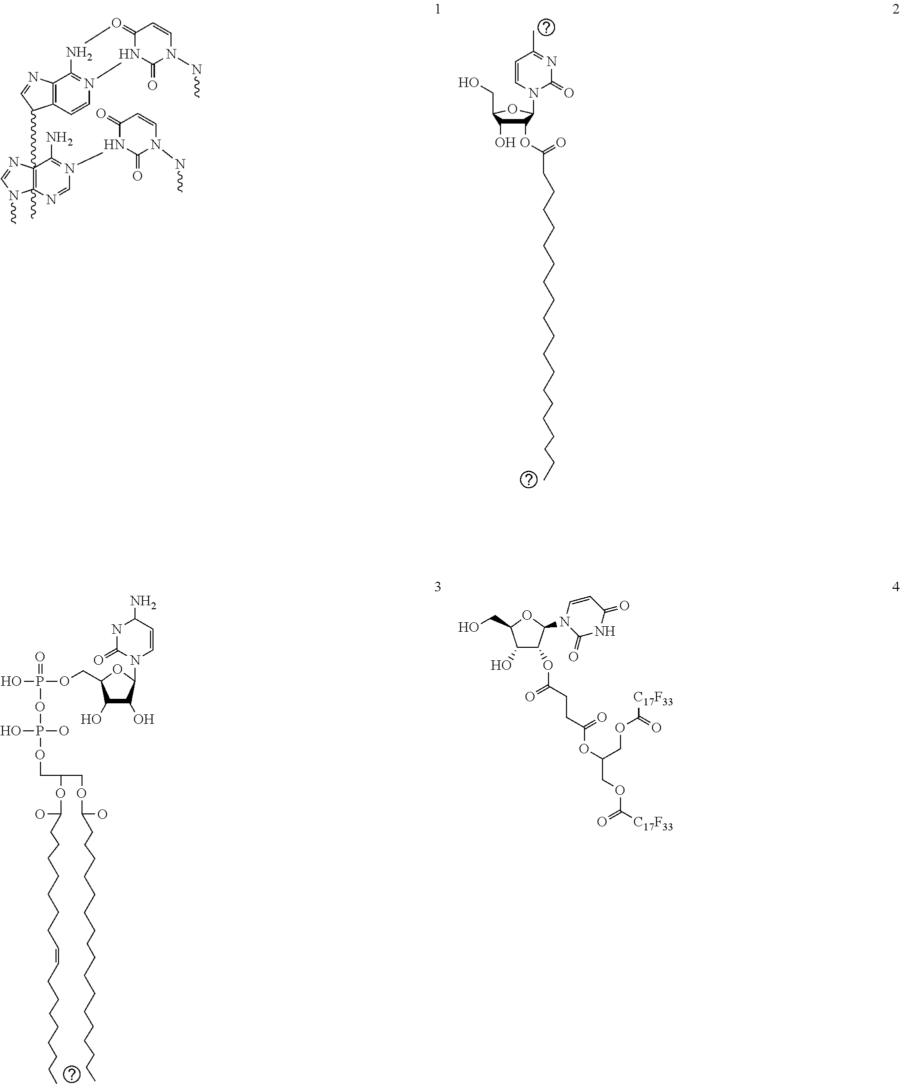

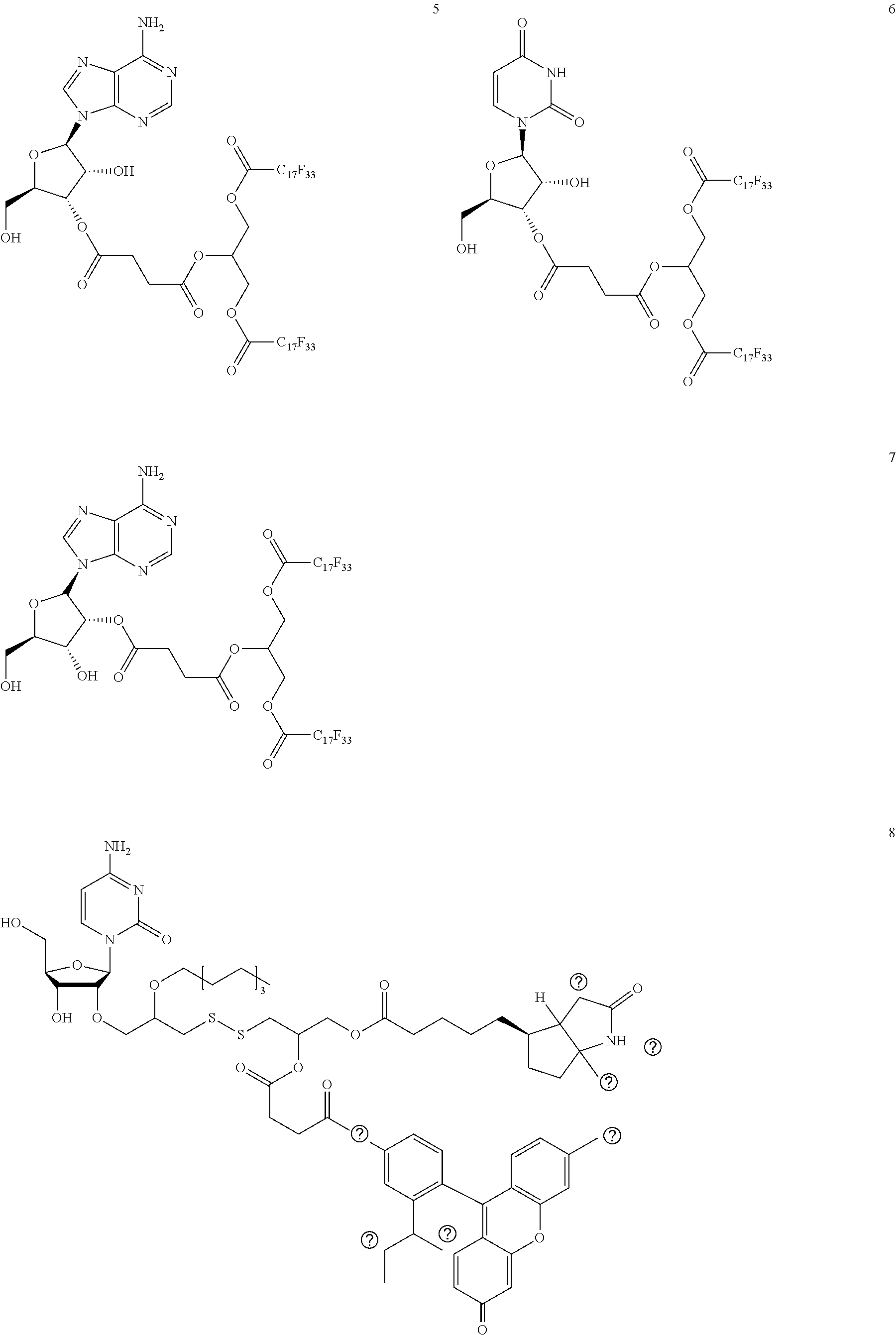

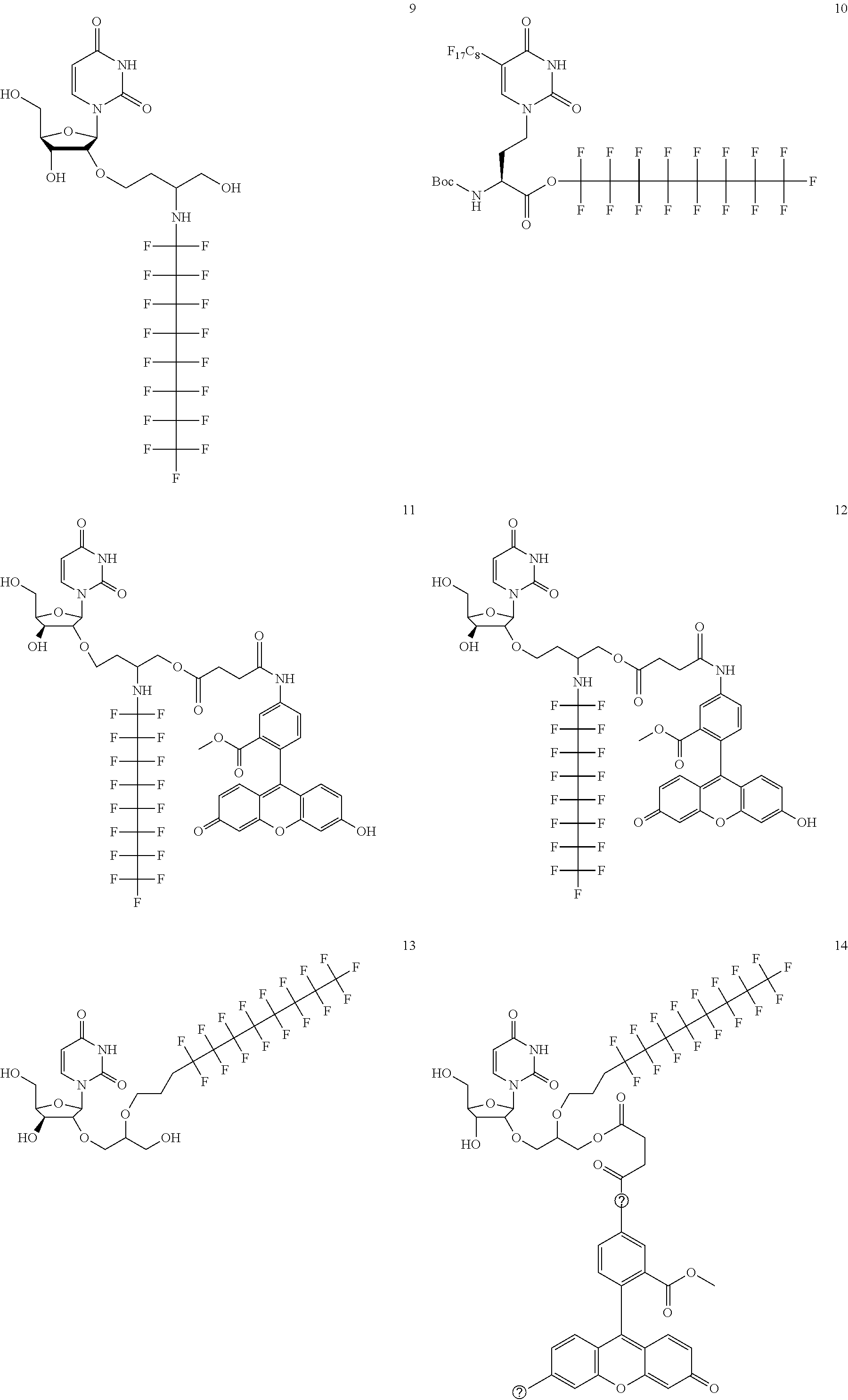

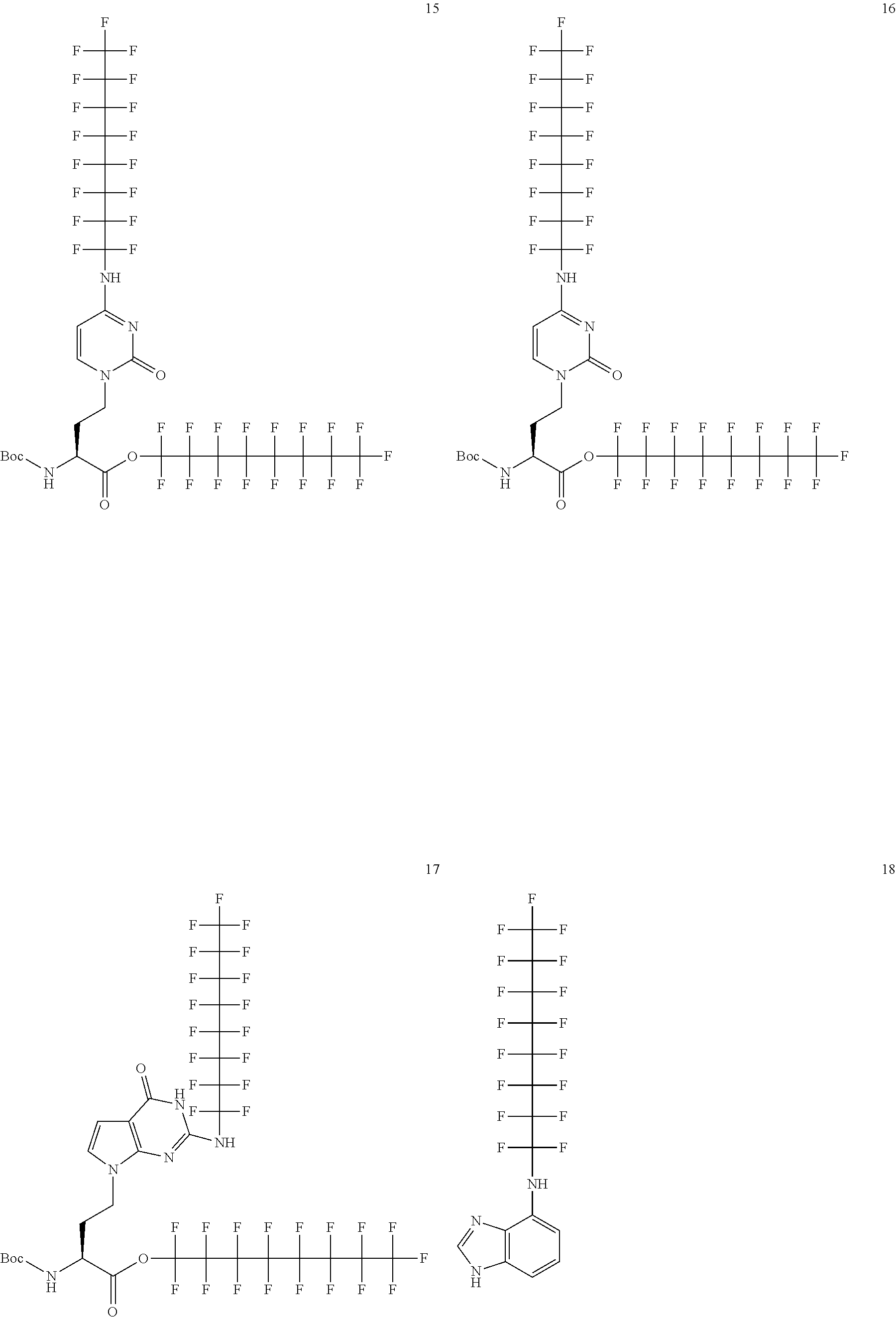

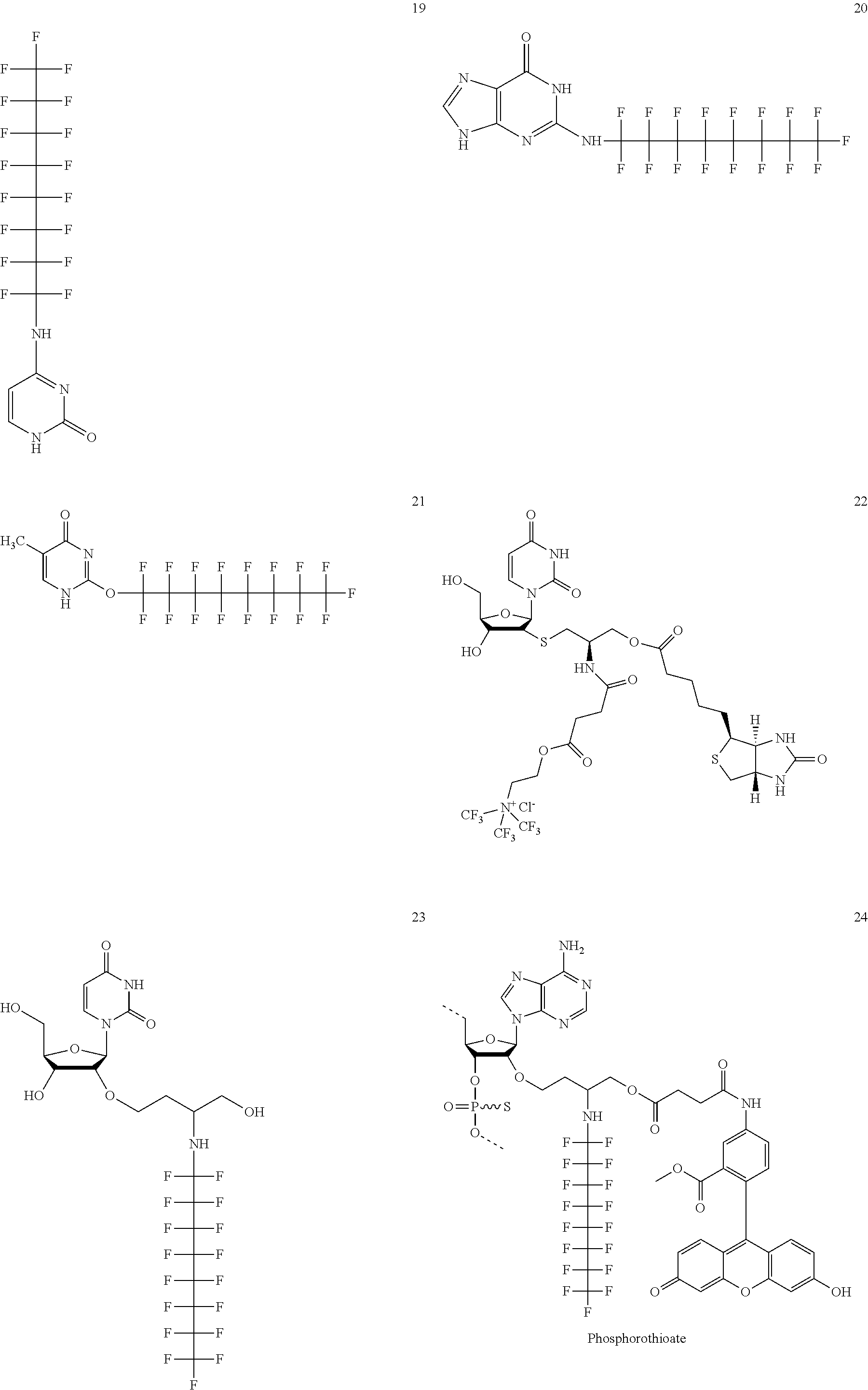

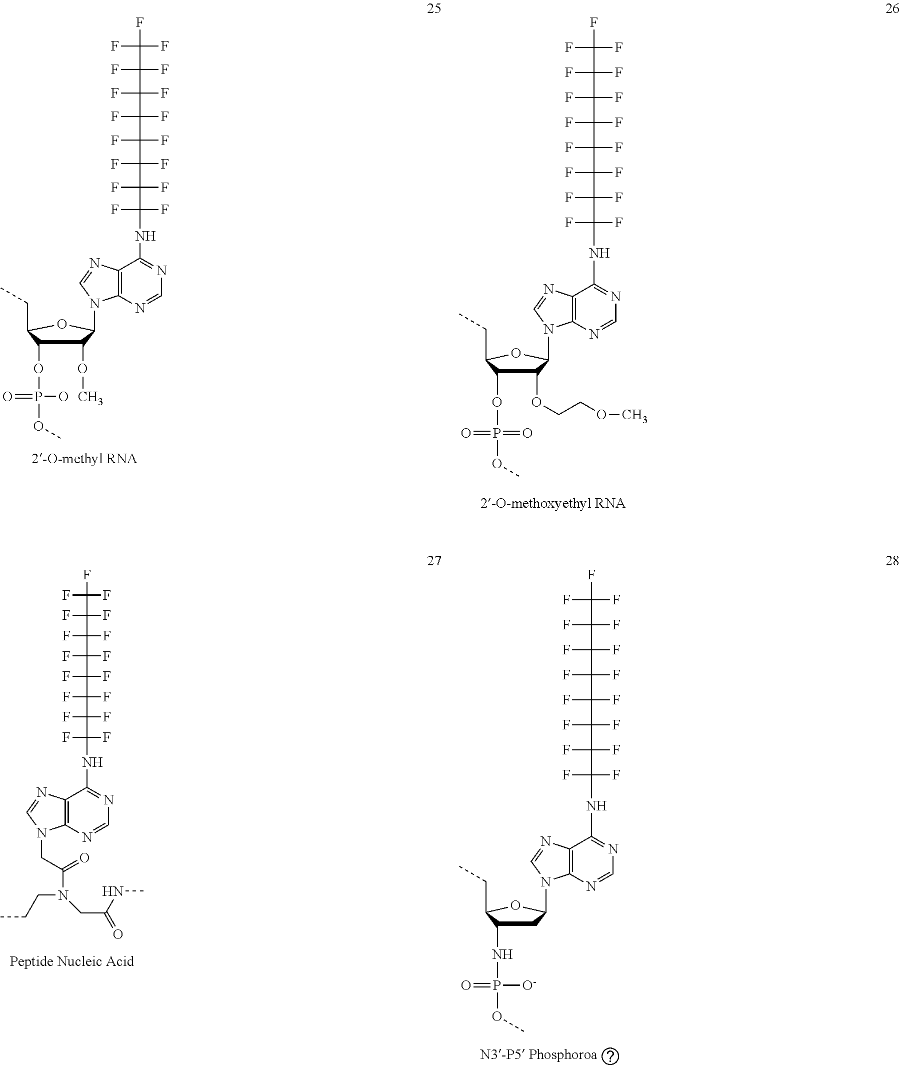

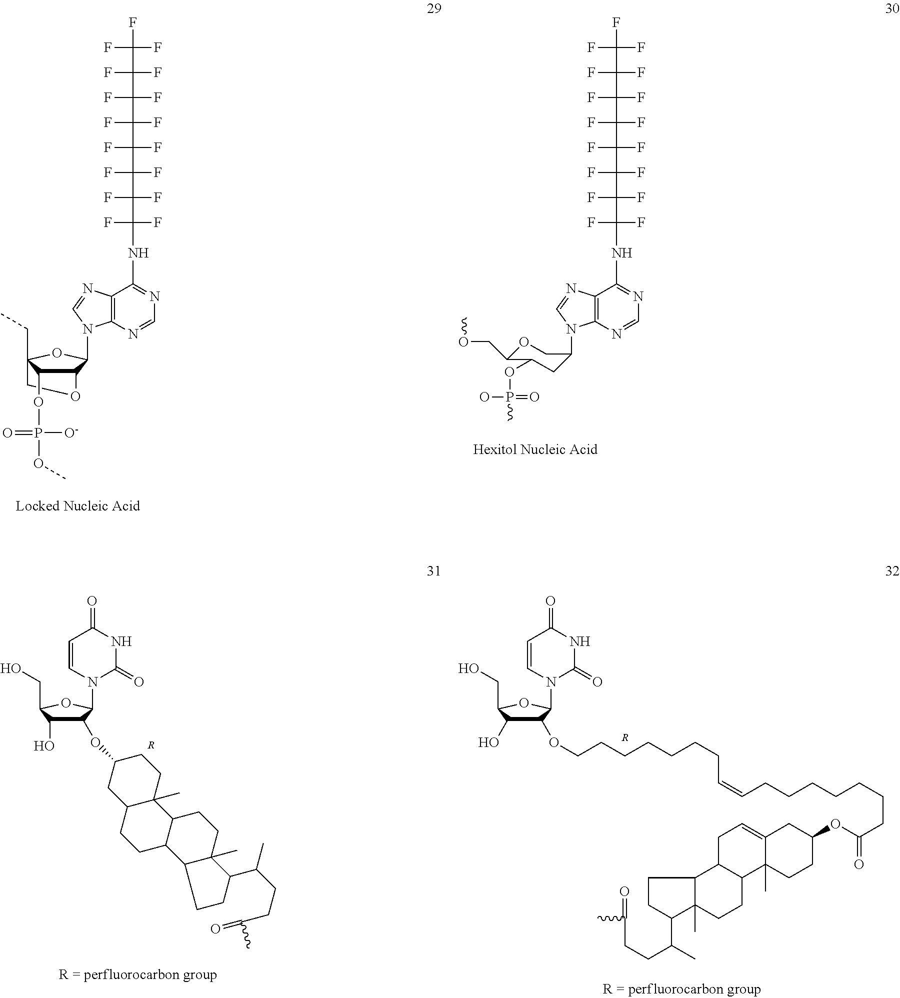

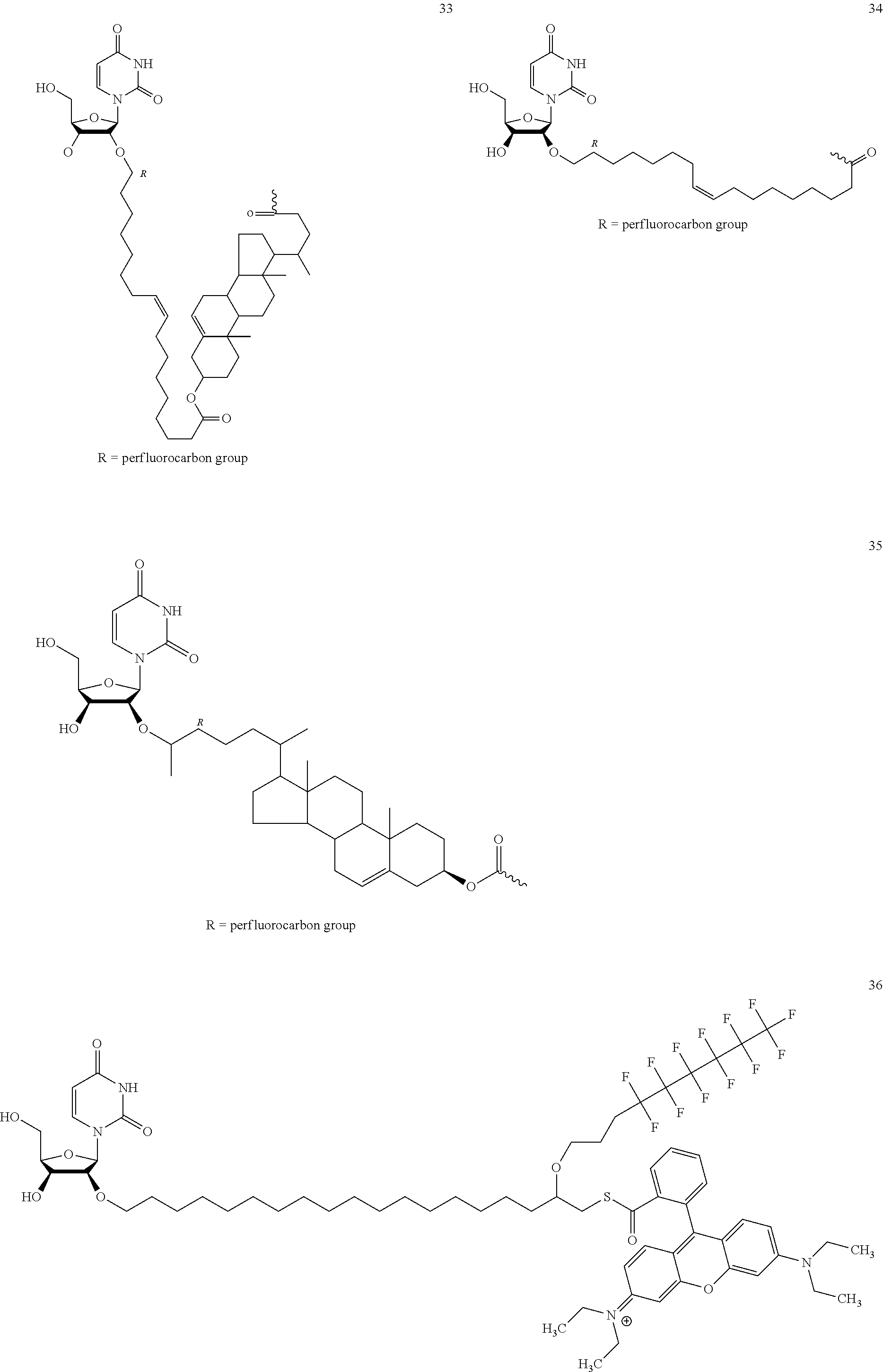

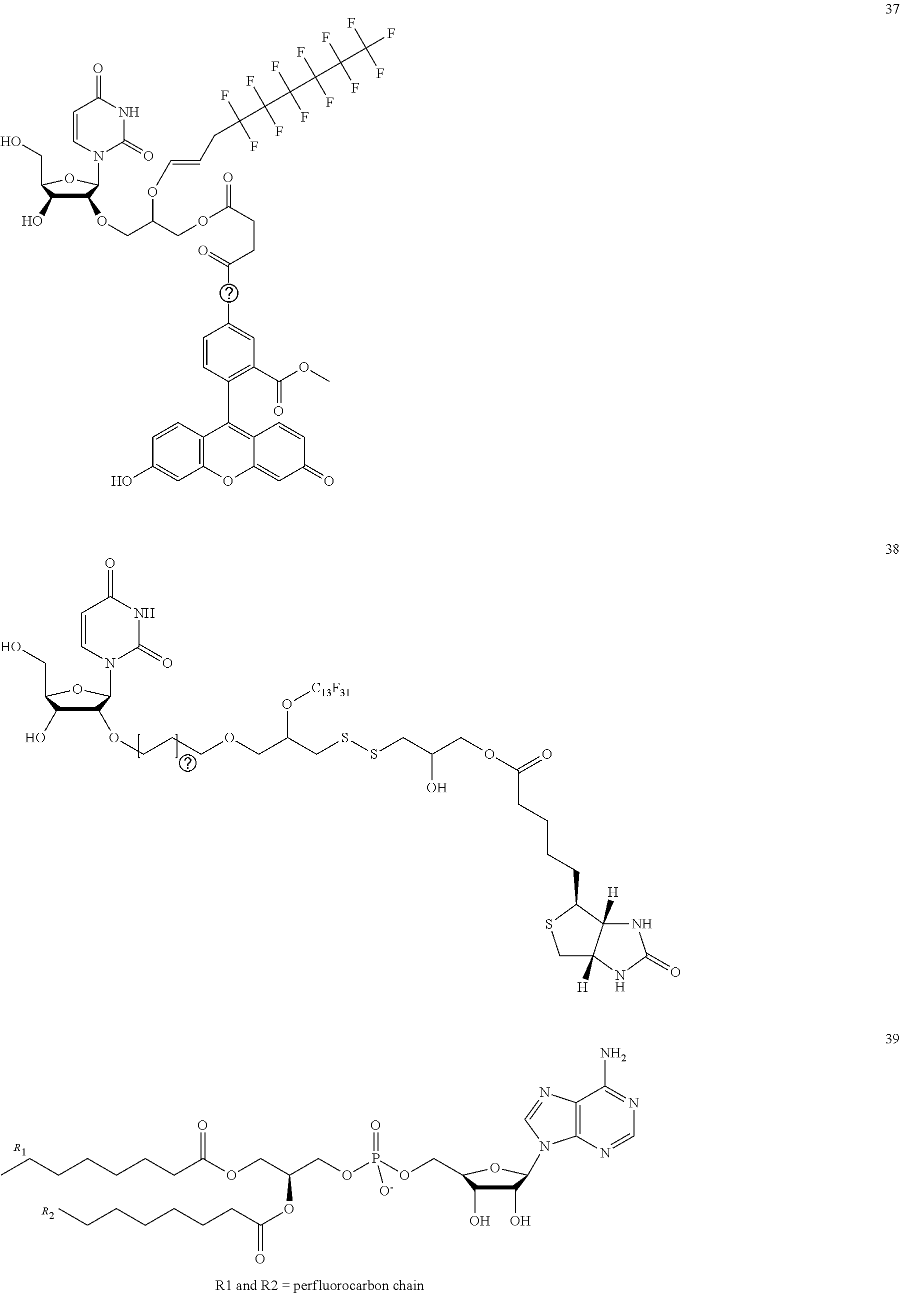

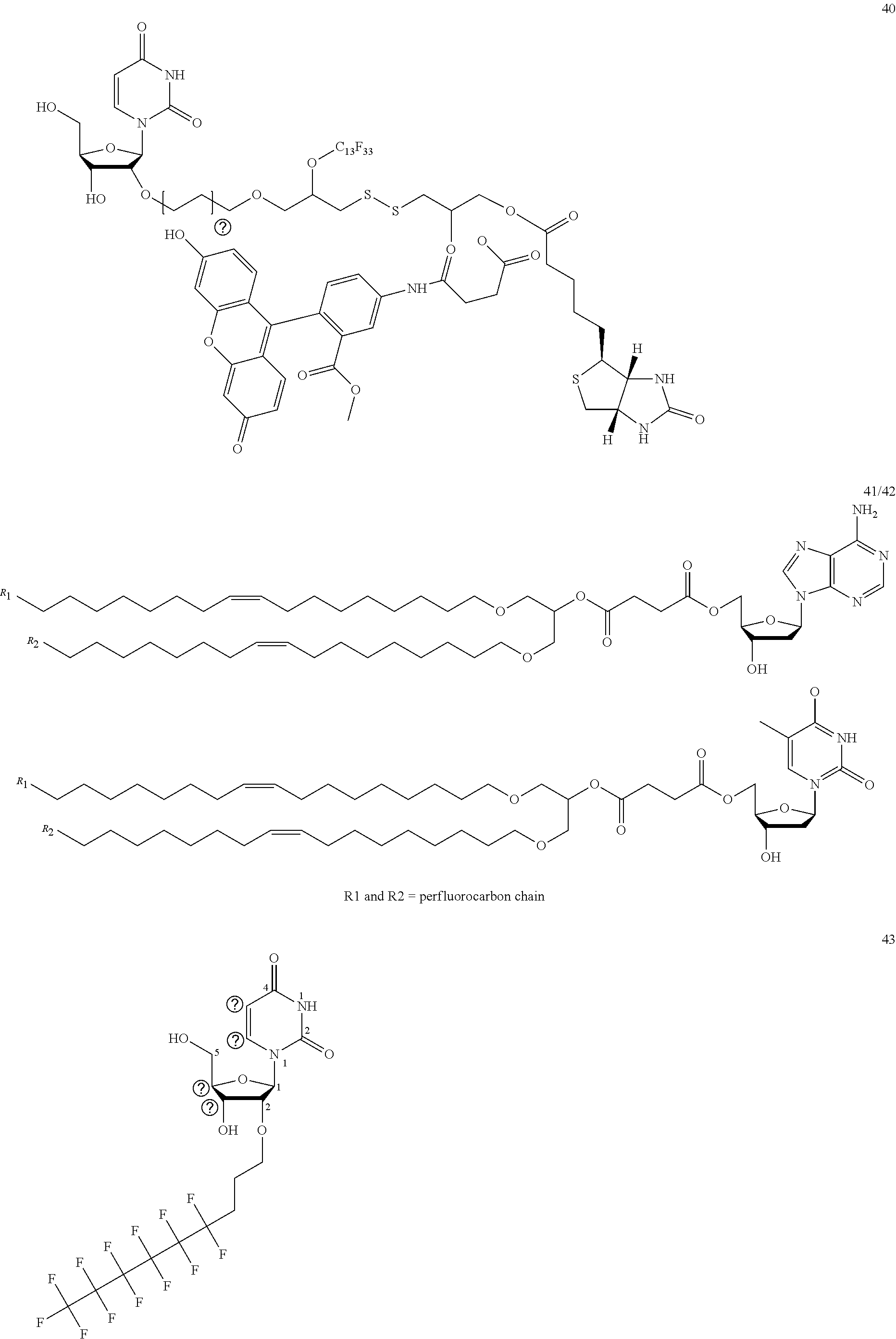

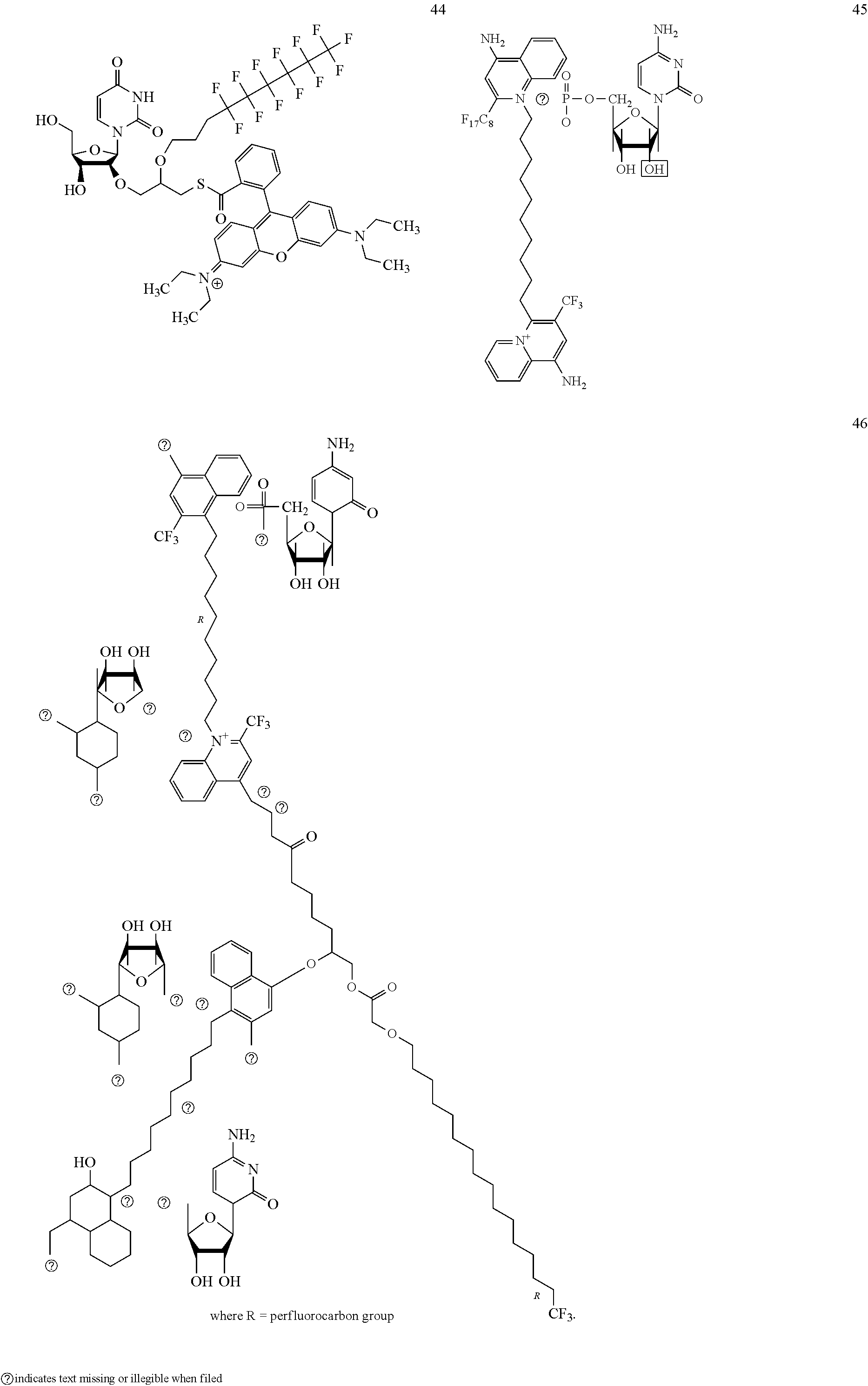

The compounds of the present method are prepared by means of known methods of synthesis which enable a successive linking of the individual molecule building blocks, for example by means of addition, substitution, condensation, etherification or esterification. Such synthesis paths are known to a synthetic chemist as a person skilled in the art.

For a better understanding of the present invention, it is explained in the following on the basis of sample embodiments with reference to the figures without being limited to these examples.

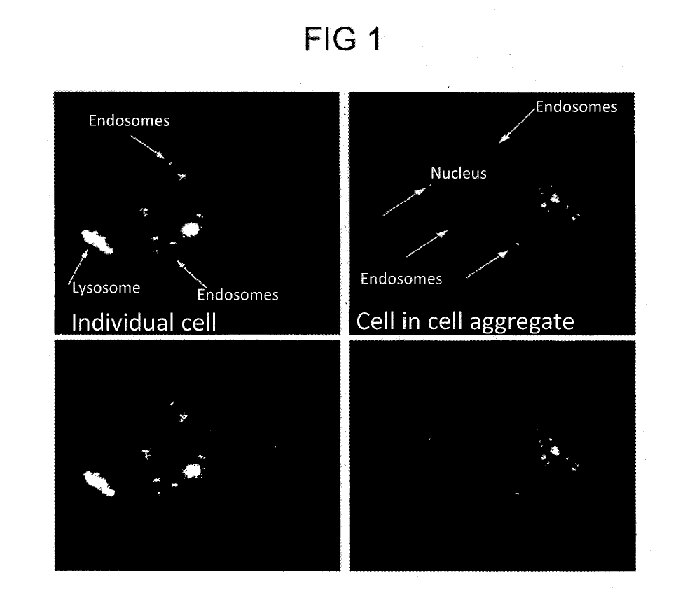

FIG. 1 shows microscopic images of cells transfected with the compounds according to the invention, and

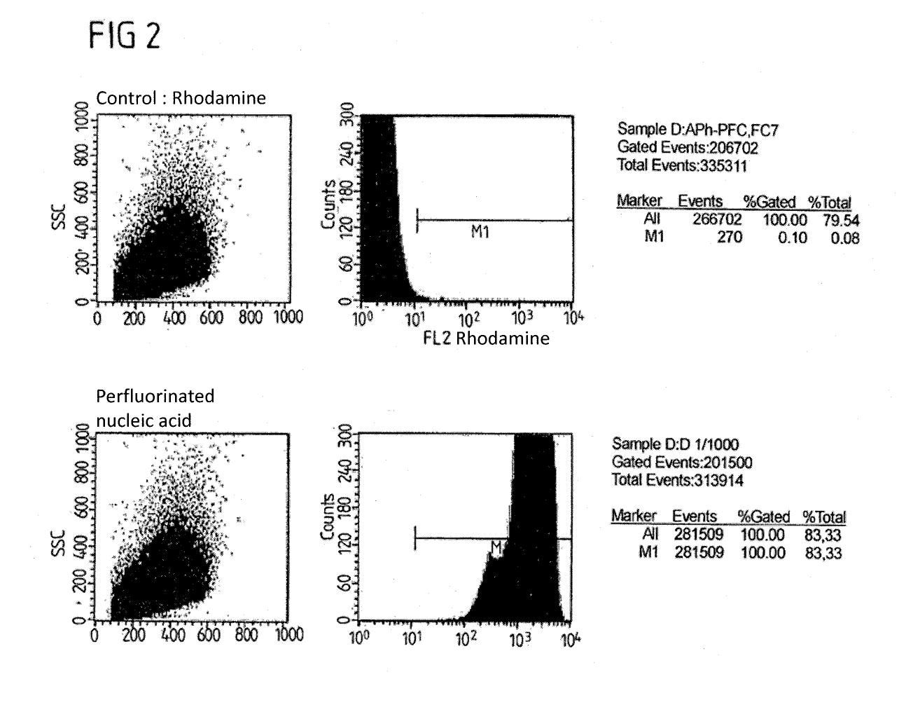

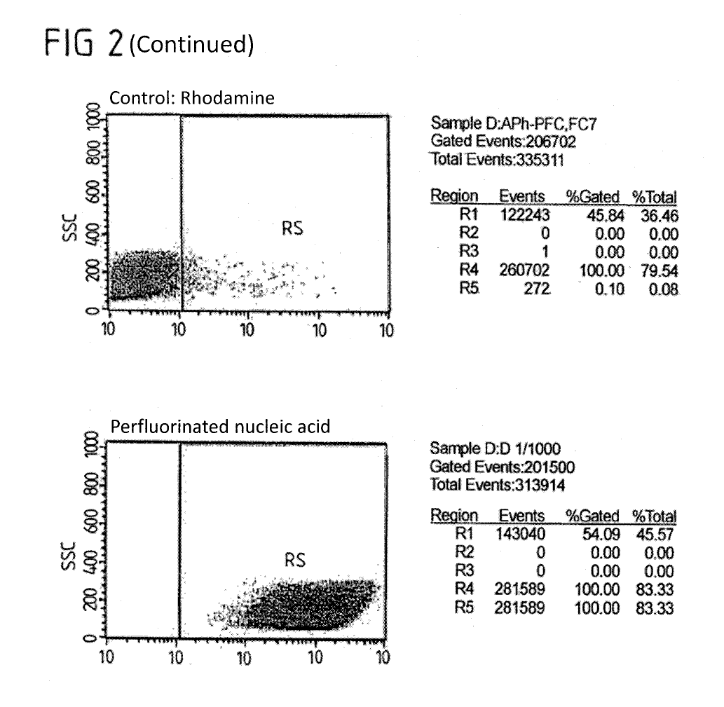

FIG. 2 shows a FACS analysis of cells transfected with the compounds according to the invention.

### SAMPLE EMBODIMENT 1

### Synthesis of Perfluorinated Nucleobases

The perfluorination of nucleobases is done by means of Williamson's ether synthesis. The NH₂ groups are relatively easily accessible here. Thymine does not have an NH₂ group but can occur in 6 tautomeric structures, of which 4 structures have a perfluorinatable OH group.

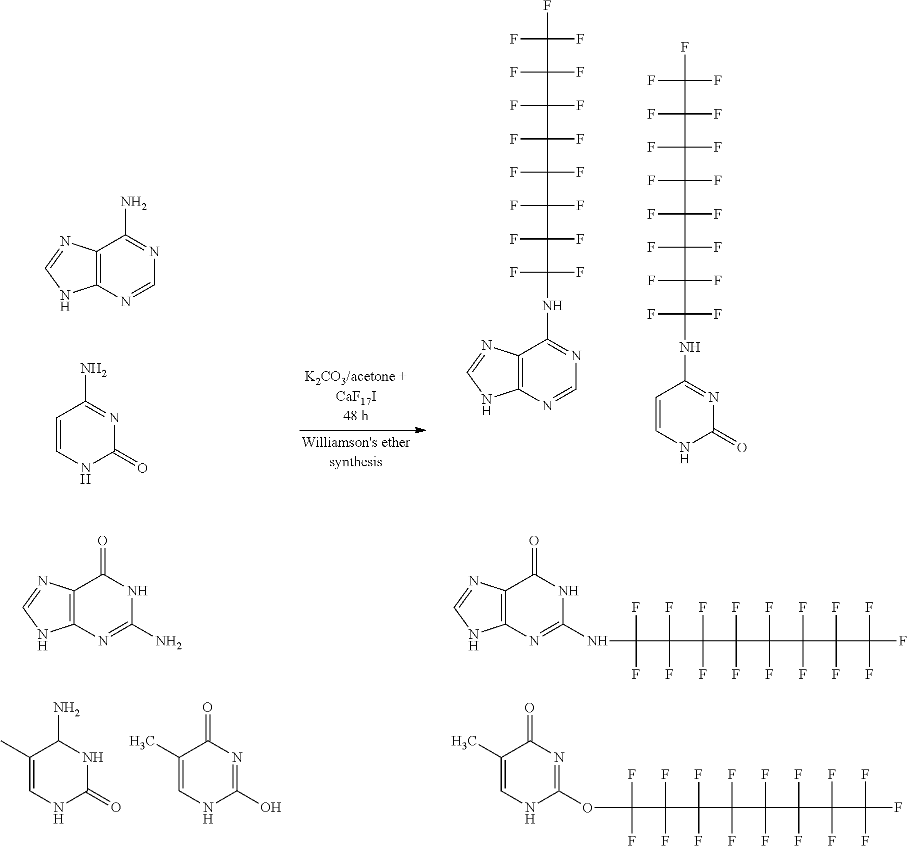

### SAMPLE EMBODIMENT 2

### Synthesis of 2′-Perfluorinated Nucleosides on the Basis of 2′-Perfluorinated Uridine

Uridine is an important component of RNA. The incorporation into RNA chain occurs via the OH groups of the 3′ and 5′ position in the sugar. The 2′ position of uridine is therefore especially suitable for the introduction of substituents without adversely affecting the bonding sites of the nucleobase. Various 2′-substituted uridines are known in which the linking occurs via 2′ ethers or esters, 2′ thioethers or esters, 2′ acid amides or 2′ carbamates or even via 2-C; see diagram 8.

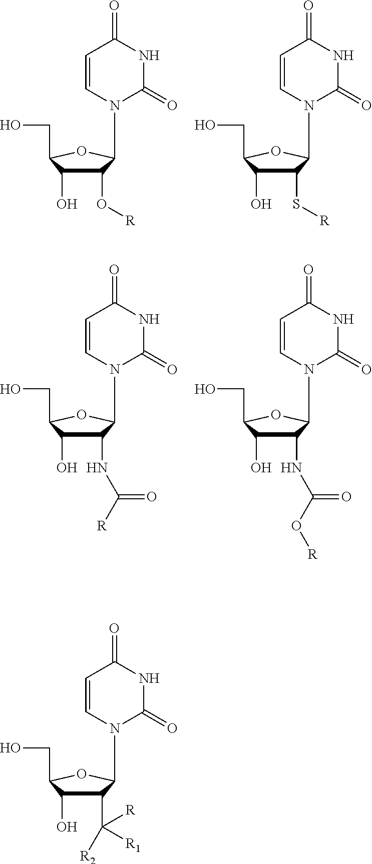

### Diagram 8: Examples of 2-Substituted Uridines Suitable for Perfluorination in 2′ Position

For the synthesis of uridines with perfluoroalkylene in the 2′ position, new syntheses have been worked out. For instance, the direct linking to the 2′-OH group is done via an ether function and an ester function; see diagram 9.

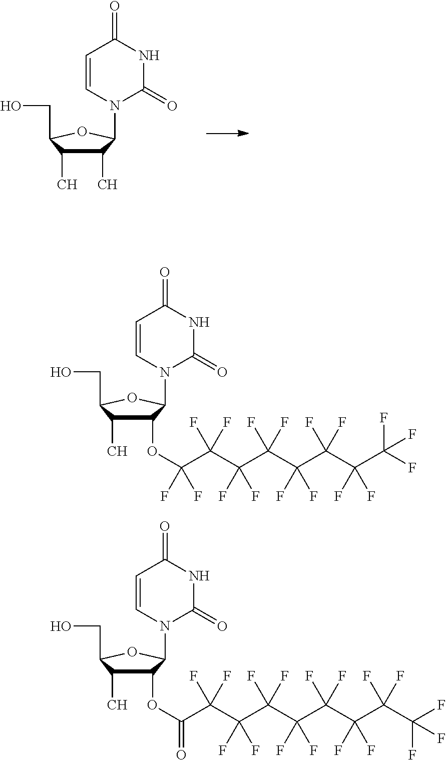

### Diagram 9: Direct Linking to the 2′-OH Group Via an Ether Function and an Ester Function

The linking to the 2′-OH group is also possible via an ether function or an ester function using a short spacer; see diagram 10.

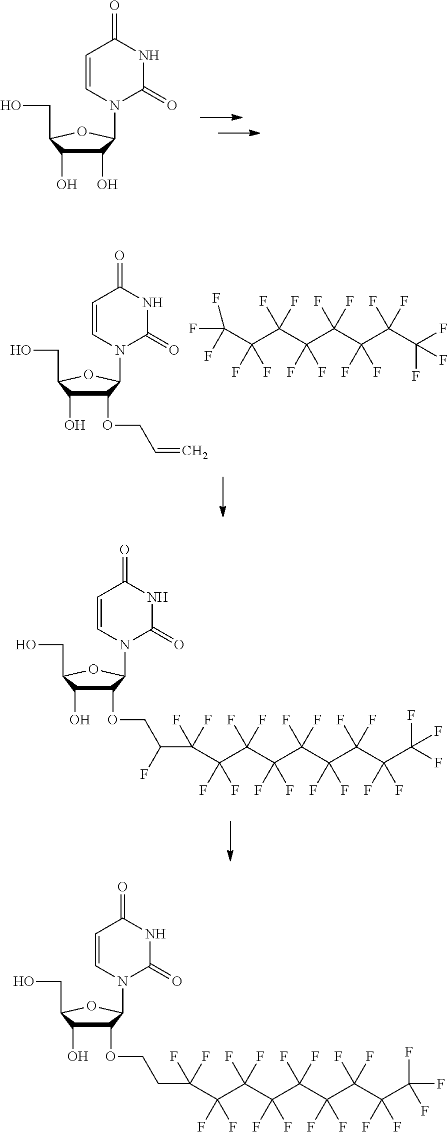

### Diagram 10: Linking to the 2′-OH Group Via an Ether Function or an Ester Function Using a Short Spacer

Another possibility is the linking of the perfluorinated alkyl group via a spacer and a predetermined breaking point; see diagram 11.

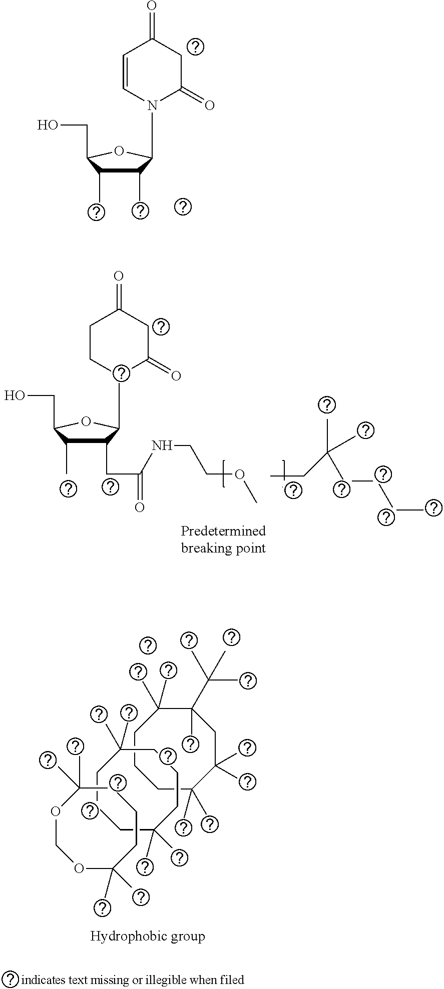

### Diagram 11: Linking of the Perfluorinated Alkyl Group Via a Spacer and a Predetermined Breaking Point

For all of the reactions depicted in diagrams 9 to 11, the 3′ and 5′-OH groups have to be protected. Silyl protective groups are suitable for this purpose. However, it must be noted here that an equilibrium between the 2′- and 3′-substituted uridine can be created in some cases (acyl migration). For this reason, the linking of the hydrophobic group to the 2′ position of the uridine was done via an ether function or via an amino function (2′-amino-2′-deoxyuridine).

Perfluorination via a 2′ ether function:

The subsequent introduction of the perfluorinated hydrophobic groups is done via the 2′-OH group via an ether function. For this, the OH groups were protected in the first step in 3′ and 5′ using tetraisopropyl-dichor-disilane, and the OH group was subsequently etherified in the 2′ position with 1-iodine-perfluoroctane or 1-iodine-perfluorine-undecane and deprotected in the next step. Depending on the course of reaction, the intermediate and end products of the reaction were purified by means of preparative chromatography. The reactions were carried out under the exclusion of moisture and under inert gas (argon). The solvents used had to also be dried before being used.

Specifically, one begins with a functionalized perfluorohydrocarbon (C₈F₁₇Br or C₈F₁₇J) and a uridine, with the 3′-OH and 5′-OH groups on the uridine first being provided with protective groups (3′,5′-dietherbutylsiloxane). The reaction with the functionalized perfluorohydrocarbon takes place at the 2′-OH group of the uridine. For this purpose, C₈F₁₇Br (or C₈F₁₇J) is bound to the 2′-OH group of the uridine by means of Williamson's ether synthesis, thus yielding uridine-2′0-C₈F₁₇ (with 3′,5′-dietherbutylsiloxane). The reaction with the functionalized perfluorohydrocarbon takes place at the 2′-OH group of the uridine. For this purpose, a reaction according to Monokanen et al. 1991 and Monokanen et al. 1993 is carried out, thus yielding Uridin-2′0-C₈F₁₇ (with 3′,5′-dietherbutylsiloxane). Finally, the protective groups are split off; see diagram 12.

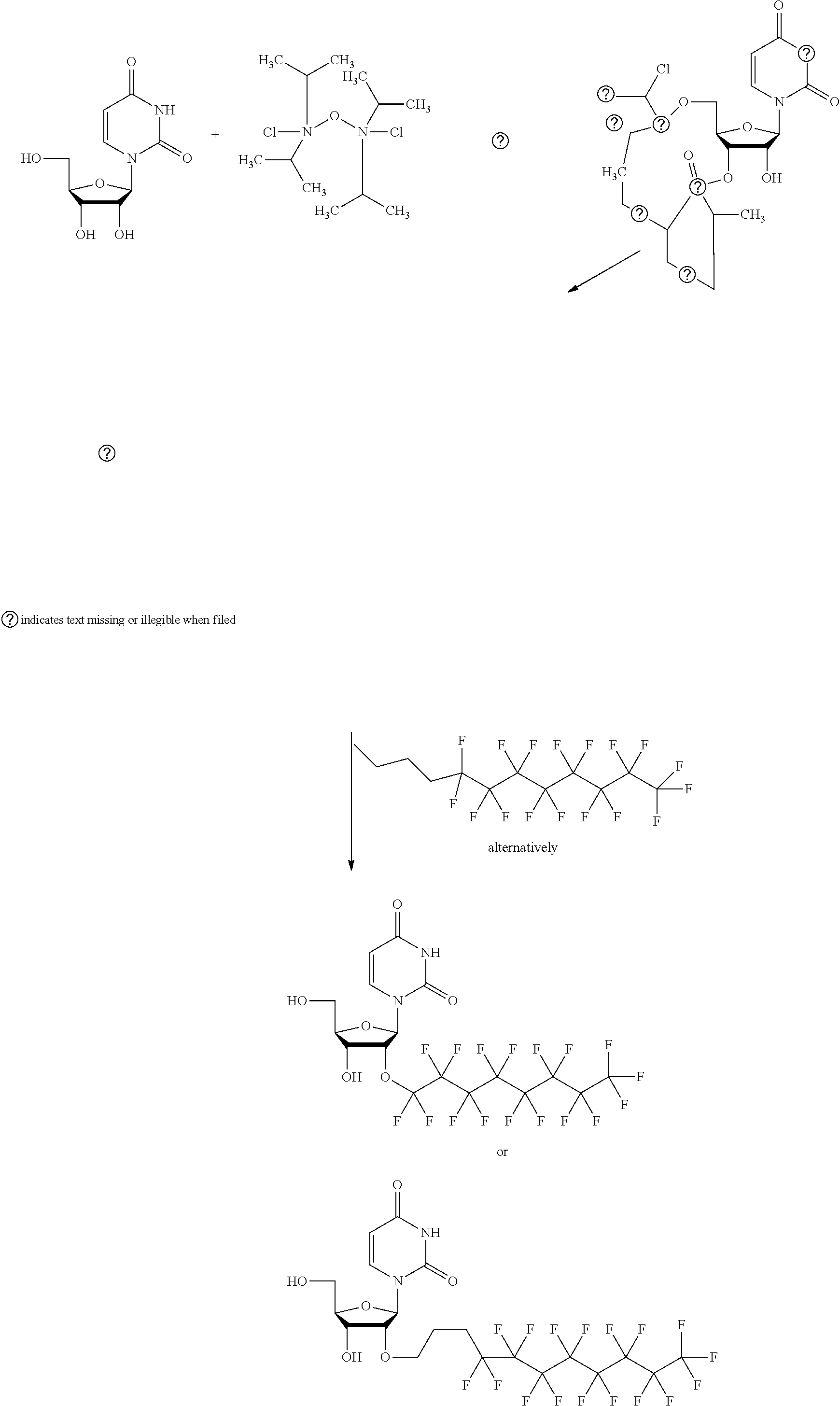

### Diagram 12

### SAMPLE EMBODIMENT 3

### Perfluorination Via a 2′-Amino Function of 2′-Amino-2′-Deoxyuridine

The second synthesis track starts with 2′-amino-2′-deoxyuridine, which is commercially available. This was converted with perfluorocarboxylic acids into acid amide. Here, too, inert gas was used under the exclusion of moisture. The end product was purified using preparative column chromatography. The primary OH group in the 5′ position of the uridine was protected with DMT-. For the analogous reactions at other positions of the nucleic acid components, adequate protective groups can be added or omitted; see diagram 13.

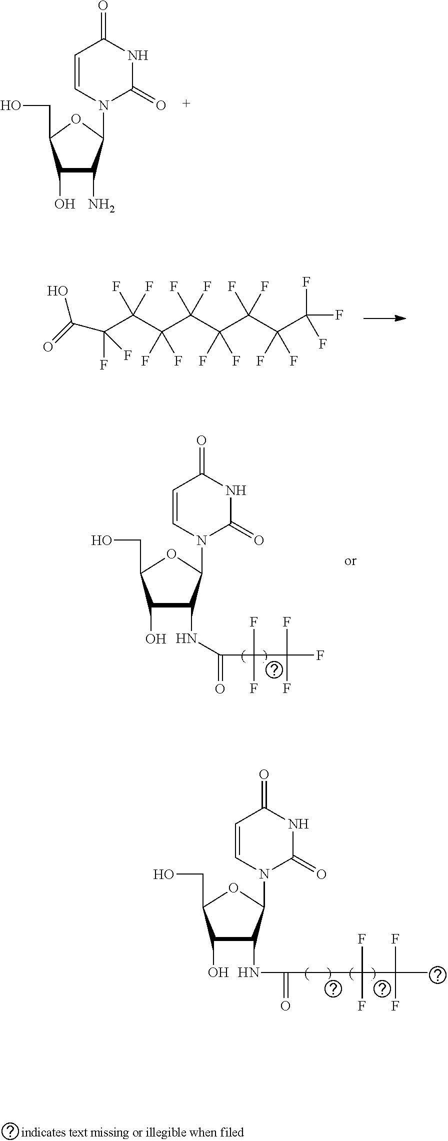

### Diagram 13

### SAMPLE EMBODIMENT 4

### Synthesis of a Perfluorinated Nucleoside with Recognition Sequence in the Form of Biotin

The starting point of the synthesis was 2′-modified uridine nucleoside with amino alcohol. The amino alcohol was perfluorinated at its amino function with C₈H₁₇OH. Through the use of 1-ethyl-3-(3-dimethylaminopropyl)carbodiimide (EDC), hydroxybenzotriazole (HOBt) and diisopropylethylamine (DIPEA), chemoselective amide formation was achieved without an attack on the alcoholic hydroxyl group. The remaining primary hydroxyl function was then esterified with biotin.

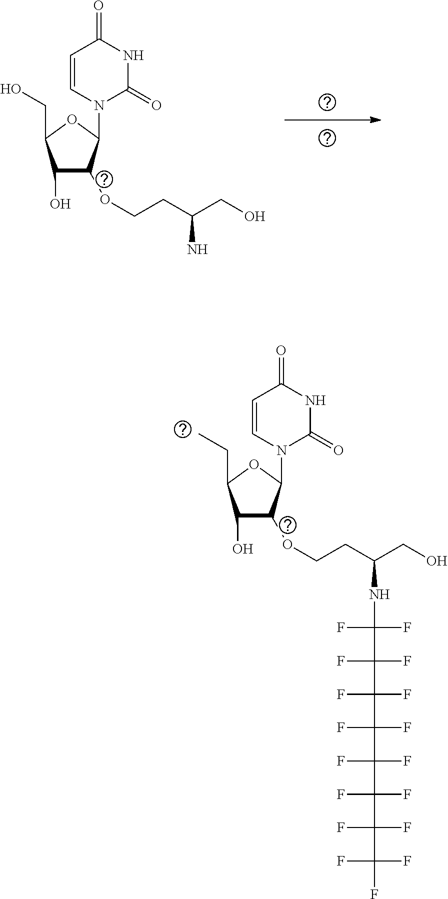

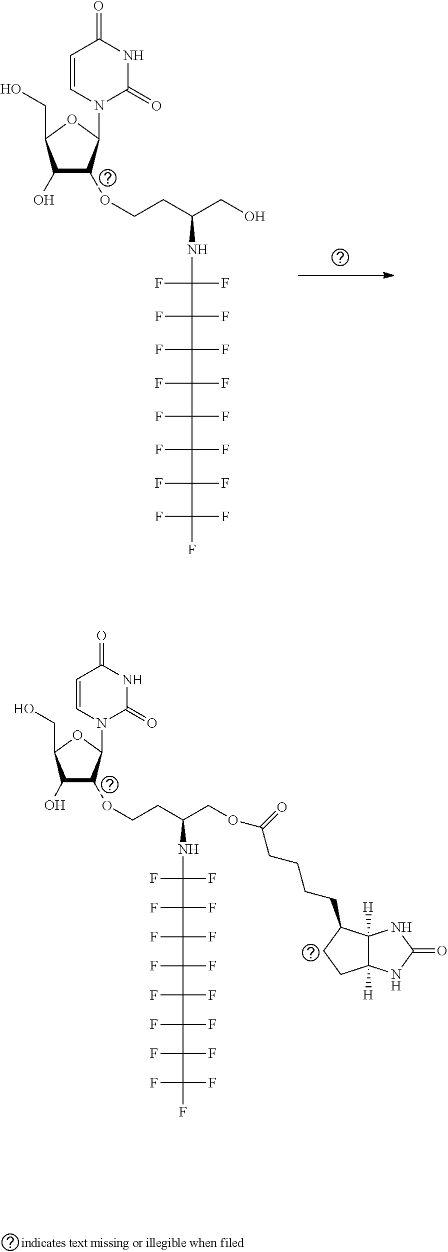

### SAMPLE EMBODIMENT 5

### Synthesis of a Perfluorinated Nucleoside with Fluorescent Dye

A fluorescein building block was synthesized with a reactive amino function, thus enabling linking via an amide bond.5-nitrofluoroscein was prepared from 4-nitrophthalic acid and resorcinol. The condensation reaction yielded a mixture of a 6- and 5-nitrofluoroscein, and the 6-nitrofluoriscein was separated off through fractional crystallization. During the subsequent synthesis, the nitro function was reduced to the amino function. The carboxyl group in the compound was converted into a methyl ester. The resulting compound was acylated with succinic acid anhydride, yielding the corresponding amide.

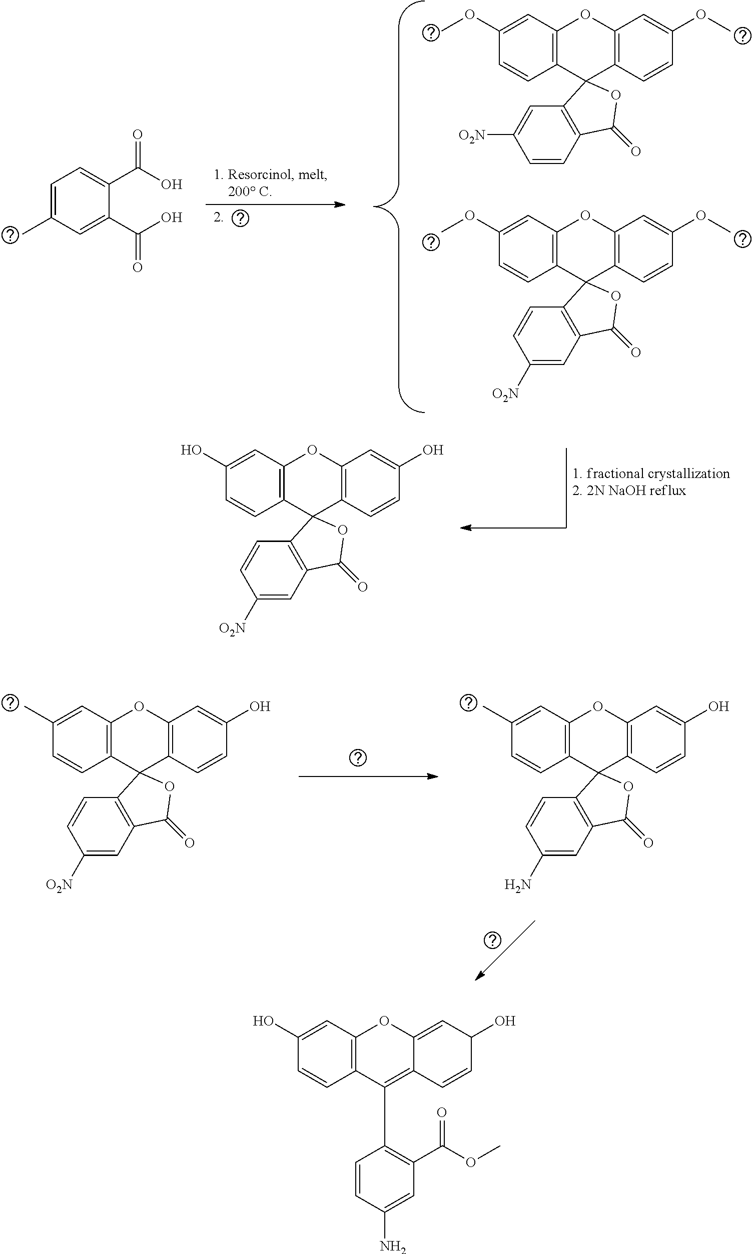

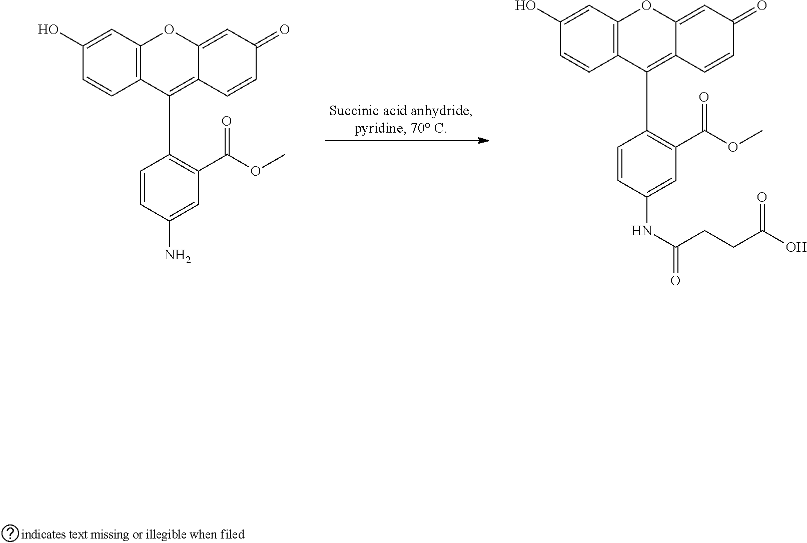 [SMILES: COC(=O)c1cc(N)ccc1-c1c2ccc(=O)cc-2oc2cc(O)ccc12 ; COC(=O)c1cc(NC(=O)CCC(=O)O)ccc1-c1c2ccc(=O)cc-2oc2cc(O)ccc12]

The free carboxyl function was esterified with a hydroxyl function of the perfluorinated nucleotide.

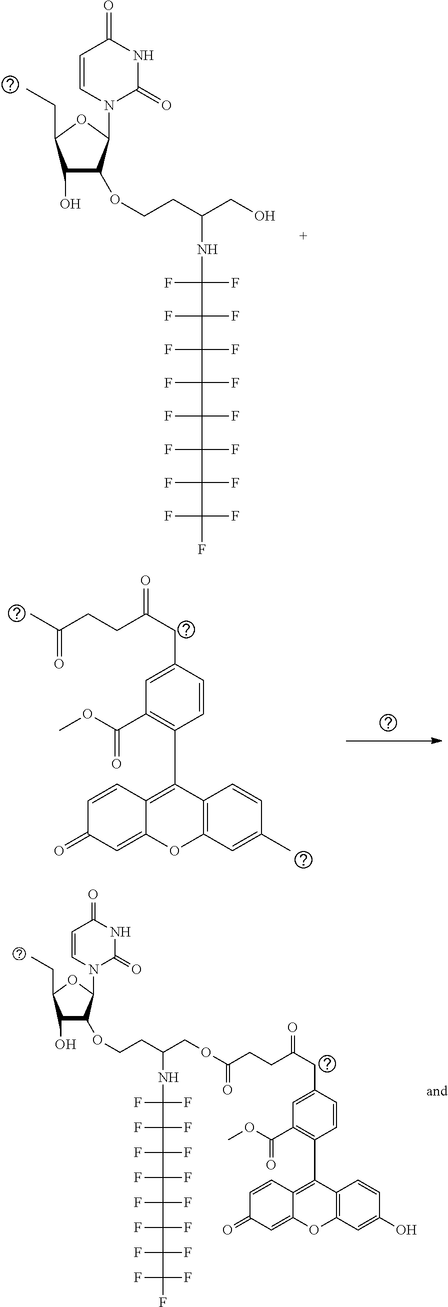

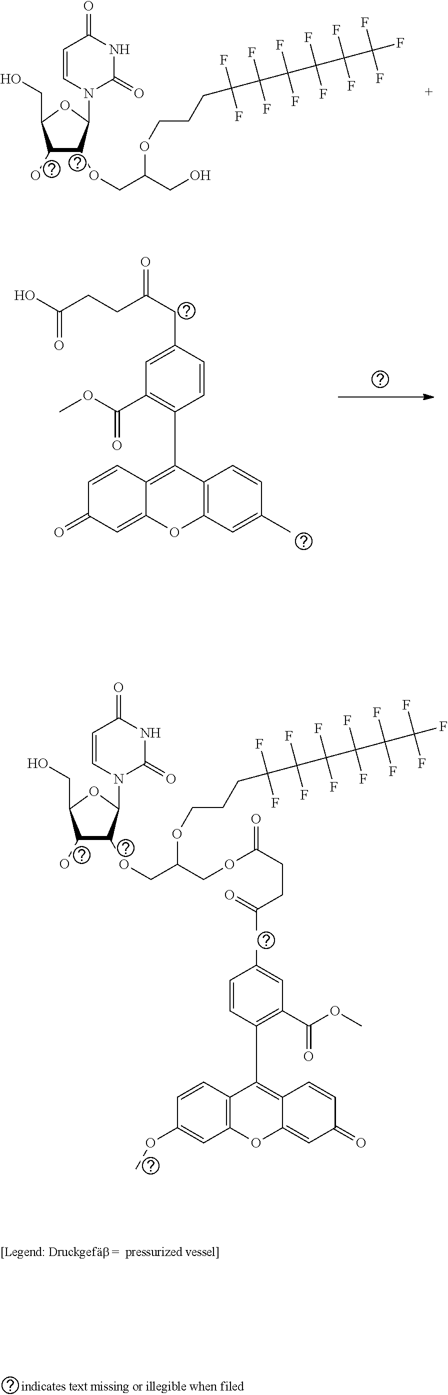

### SAMPLE EMBODIMENT 6

### Synthesis of a Perfluorinated Nucleoside with Recognition Sequence and Fluorescent Dye

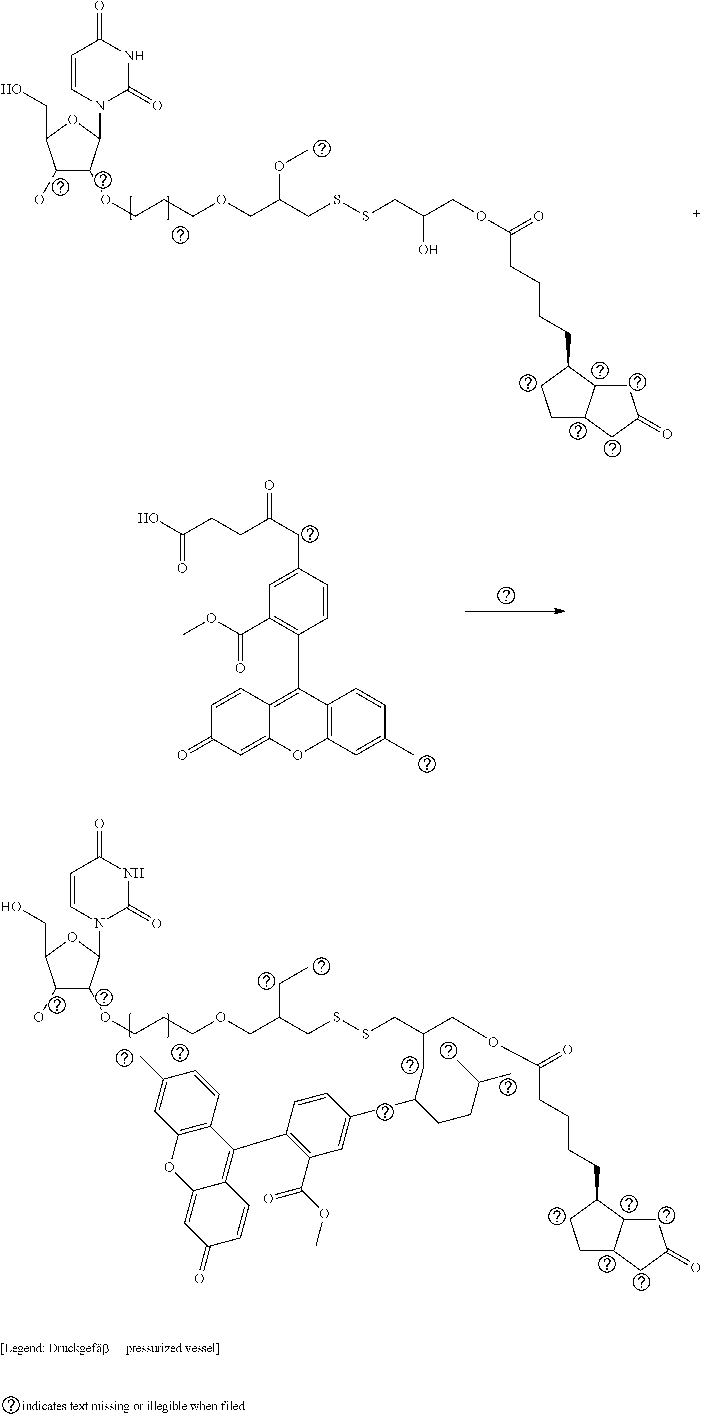

### SAMPLE EMBODIMENT 7

### Synthesis of Perfluorinated Oligonucleotides and Nucleic Acids

In a baked-out 50 ml Schlenk tube, 0.90 mmol 2′-perfluorinated uridine is dissolved in 20 ml dry dichloromethane under an argon atmosphere. After adding 1.00 mmol CyTIPP and 2.30 ml of a 0.45 M tetrazole solution (in acetonitrile) to the solution, the reaction mixture is stirred for 2 h at room temperature and the progress of the reaction is monitored by means of DC. After concentrating the mixture on the rotary evaporator and after column-chromatographic purification, one obtains 3′-phosphoramidite-2′-Perfluoro-uridine. These nucleotides are the starting point for the phosphoramidite method in which the monomeric nucleotides are coupled together. In this process, monomeric nucleotide building blocks are linked together as 3′-phosphoramidites to a solid phase. The construction of the sequence occurs from the 3′ to the 5′ end, with a “leader nucleoside” linked in standard syntheses to a suitable solid phase via a 3′ succinate. The nucleosides brought in as 3′ phosphoramidite bear an acid-labile protective group at the 5′ hydroxyl group and base-labile protective groups at the exocyclic amino functions of the bases. The synthesis cycle, which is repeated over and over again, comprises the following reactions: Phosphoramidite is activated through the addition of a weak acid, such as 4,5-dicyanoimidazole or 1-H-tetrazole, and coupled with the 5′-hydroxy group of the immobilized nucleoside. The unconverted OH groups are acetylated with acetic acid anhydride and thus blocked from further conversions. Oxidation occurs with aqueous iodine solution and leads to phosphoric acid triester. The acid-labile trityl protective group is removed with a dichloroacetic acid solution. Upon completion of the sequence, the oligonucleotide and the protective groups of the nucleobases are cleaved off through treatment with aqueous ammoniac at 55° C.

The phosphoramidite method or other chemical methods for the preparation of oligomers and nucleic acids are already offered in the present art by various companies worldwide, and the achievable length of the nucleotide sequences has reached the size of artificial genes.

### SAMPLE EMBODIMENT 8

### Alternative Synthesis of Perfluorinated Oligonucleotides and Nucleic Acids

The perfluorination of entire oligomers and nucleic acids is also possible, for example through acid-catalyzed methods with fluoric acid, or the polymerization of perfluorinated nucleotides is possible using polymerases (Polymerase Chain Reaction).

### SAMPLE EMBODIMENT 9

### Perfluorination of Modified Nucleic Acid Building Blocks

Nucleic acids can be derivatized to stabilize or eliminate the electrical charge, for example by means of phosphorothioates, electrically neutral methyl phosphonate derivatives, electrically neutral dimethyl sulfone derivatives or dervatization at the 2′ carbon atom of the ribose. Just like with normal nucleic acid building blocks, the possibilities for perfluorination are on the modified sugar-phosphate backbone and at the bases. For perfluorination paths, see Synthesis of perfluorinated nucleosides, nucleotides and perfluorinated oligonucleotides.

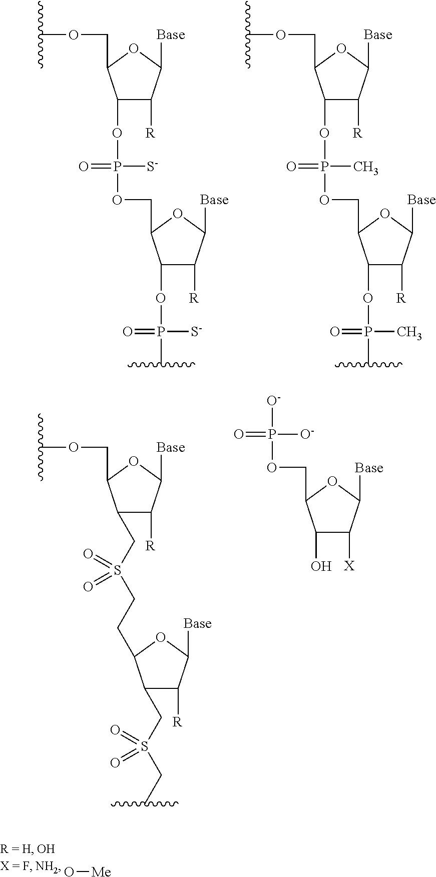

### SAMPLE EMBODIMENT 10

### Synthesis of Perfluorinated Peptide Nucleic Acid Monomers in the Form of Alanyl Nucleoamino Acids

Peptide nucleic acid monomers are nucleotide analogs in which the ribose-phosphodiester backbone has been replaced by a peptidic, achiral backbone that is based on N-(2-aminoethyl)glycine subunits or other peptide units. Here, the perfluorinated bases are linked to the backbone via a carboxymethylene unit. While perfluorination at the NH₂ groups of the nucleobases exhibits no impact on the incorporation into an oligomer, the perfluorination at the amino-terminal and carboxy-terminal ends of the peptide units has the effect of halting synthesis. The synthesis of alanyl nucleoamino acids starts from A/-Boc-L-serine and A/-Boc-D-serine that have been converted into A/Boc-L-serine lactone. The Boc-serine lactone reacts by means of nucleophilic ring opening through the benzyloxycarbonyl-protected cytosine and the guanine precursor 2-amino-6-chloropurine to Boc-L-AlaG-OH or Boc-D-AlaG-OH and Boc-AlaCZ-OH or its enantiomer. The protective group benzyloxycarbonyl at the exocyclic amino function of the cytosine is necessary for the subsequent peptide solid-phase synthesis. The guanine does not require any protection, since the exocyclic amino group exhibits very little nucleophilia.

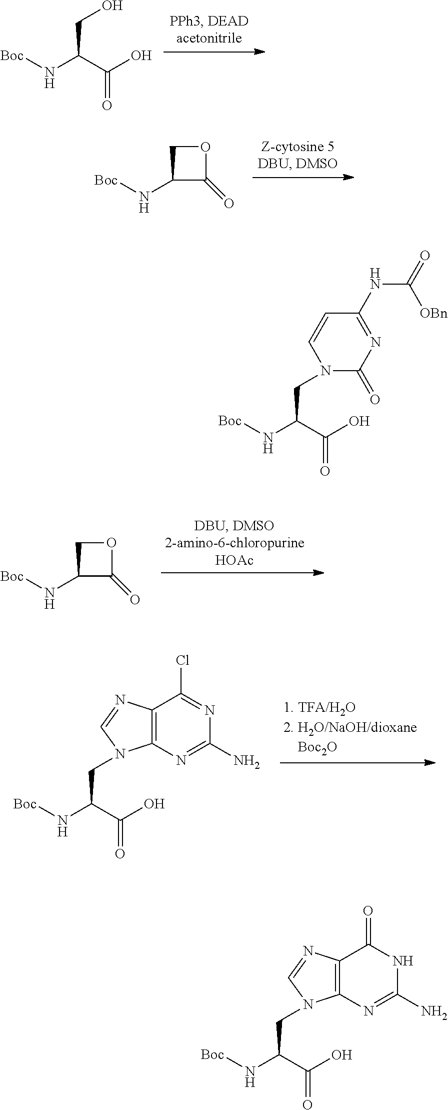 [SMILES: CC(C)(C)OC(=O)N[C@@H](CO)C(=O)O ; CC(C)(C)OC(=O)N[C@H]1COC1=O ; CC(C)(C)OC(=O)N[C@@H](Cn1ccc(NC(=O)OCc2ccccc2)nc1=O)C(=O)O ; CC(C)(C)OC(=O)N[C@H]1COC1=O ; CC(C)(C)OC(=O)N[C@@H](Cn1cnc2c(Cl)nc(N)nc21)C(=O)O ; CC(C)(C)OC(=O)N[C@@H](Cn1cnc2c(=O)[nH]c(N)nc21)C(=O)O]

A/-Boc-L-asparaginic acid benzyl ester or Boc-D-asparaginic acid benzyl ester was used as the starting compound for the synthesis of homoalanyl-nucleoamino acids. The side chains were reduced with BH3-THE to alcohol and brominated via an Appel reaction into N-Boo-D-γ-bromine-homoalanyl benzyl ester or N-Boc-D-Y-bromine-homoalanyl benzyl ester.

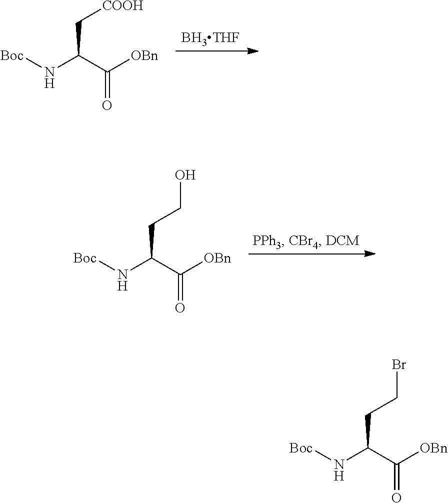

Under K₂CO₃, a nucleophilic substitution of the bromide with benzyloxycarbonyl-protected cytosine and 2-amino-6-chloropurine was performed. In the next step, TFA/H₂O was hydrolysed, with the Boc protective group being removed simultaneously. The subsequent step was carried out hydrogenolytically with PdO-H₂O. This was followed by protection with Boc anhydride into Boc-L-HalG-OH or Boc-D-HalG-OH.

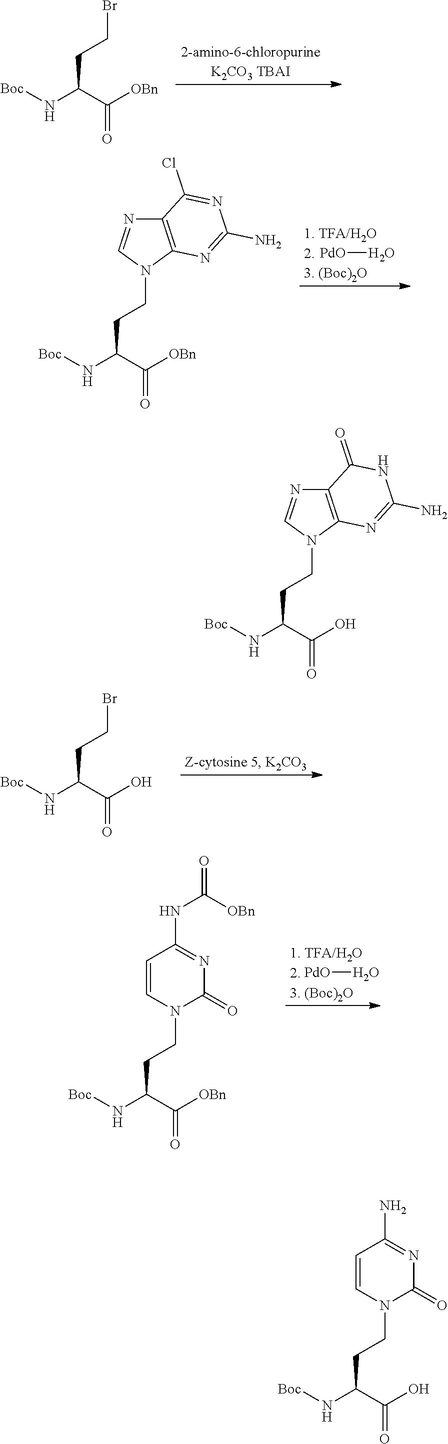

The perfluorination of the monomers is done by means of Williamson's ether synthesis using K₂CO₃/acetone and a functionalized perfluorocarbon molecule over 48 hours. All of the accessible NH₂ and OH groups at the nucleobases and at the peptide building blocks were perfluorinated. One example of this perfluorination is shown here using the example of C₈F₁₇J.

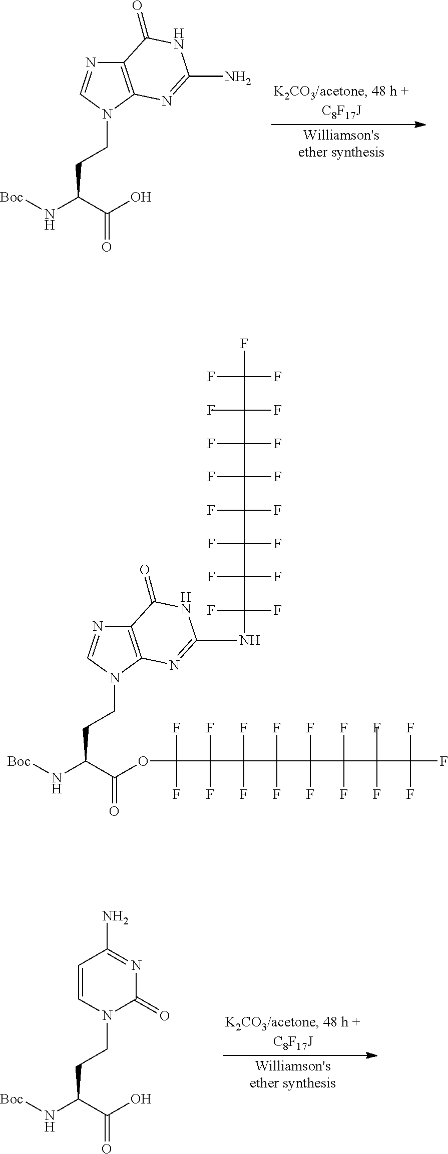

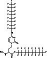

Another possibility is the masking of the OH groups of the peptide fraction before perfluorination takes place. Under those circumstances, only the NH₂ groups of the nucleobases der Nukleobasen are perfluorinated. To achieve this, the hydroxy groups must be protected with ditertbutylsilylditriflate. After perfluorination, these OH groups must be deprotected again in order to be available for peptide synthesis. Using these monomers, an oligomer synthesis is possible in which perfluorinated monomers are already incorporated. However, due to the additional reaction steps that are required, it appears to be easier to first synthesize and then perfluorinate the oligomer.

Additional perfluorinations listed for the sake of example:

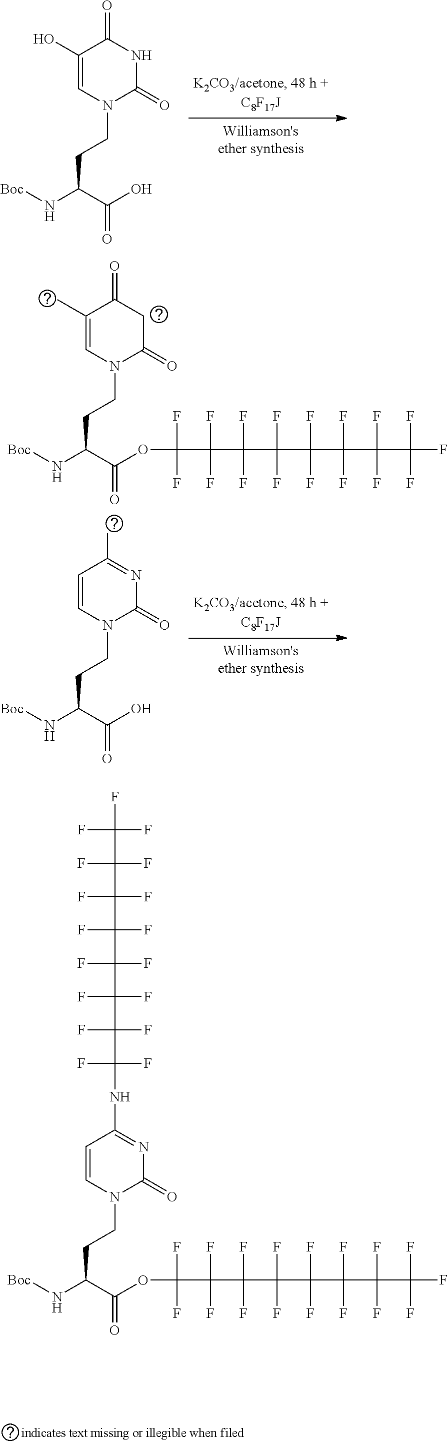

### SAMPLE EMBODIMENT 11

### Synthesis of Perfluorinated Peptide Nucleic Acid Oligomers and Peptide Nucleic Acids

In the case of peptide nucleic acids, the entire ribose-phosphodiester backbone was replaced by a peptidic, achiral backbone based on N-(2-aminoethyl)glycine subunits or other peptide units. The perfluorinated bases were linked here to the backbone via a carboxymethylene unit. Next, the nucleobases were linked to the peptide units. Then the monomers were linked to oligomers in solid-phase synthesis. It has proven simpler to perform the perfluorination steps only after the synthesis of the oligomers. The perfluorination steps follow the reaction steps used in the perfluorination of the monomers.

The synthesis of alanyl-nucleoamino acids starts from N-Boc-L-serine and A/-Boc-D-serine that have been converted into N-Boc-L-serine lactone or N-Boc-D-serine lactone. The Boc-serine lactone reacts by means of nucleophilic ring opening through the benzyloxycarbonyl-protected cytosine and the guanine precursor 2-amino-6-chlorepurine into Boc-L-AlaG-OH or Boc-D-AlaG-OH and Boc-AlaCZ-OH or its enantiomer. The protective group benzyloxycarbonyl on the exocyclic amino function of the cytosine is necessary for the subsequent peptide solid-phase synthesis. The guanine does not require any protection, since the exocyclic amino group exhibits very little nucleophilia.

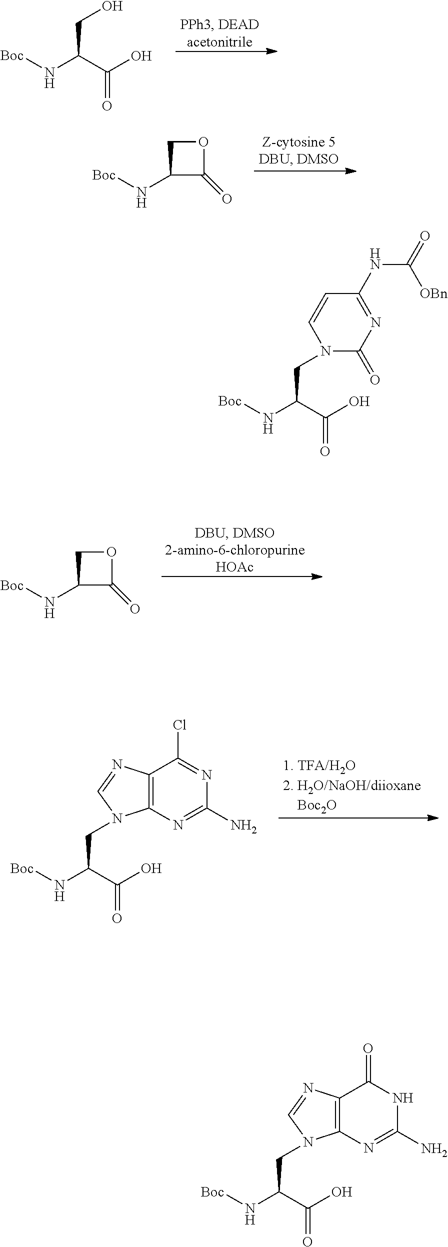

A/-Boc-L-asparaginic acid benzyl ester or Boc-D-asparaginic acid benzyl ester was used as the starting compound for the synthesis of homoalanyl-nucleoamino acids. The side chains were reduced with BH3-THF to alcohol and brominated via an Appel reaction into N-Boc-L-γ-bromine-homoalanyl benzyl ester or N-Boc-D-γ-bromine-homoalanyl benzyl ester.

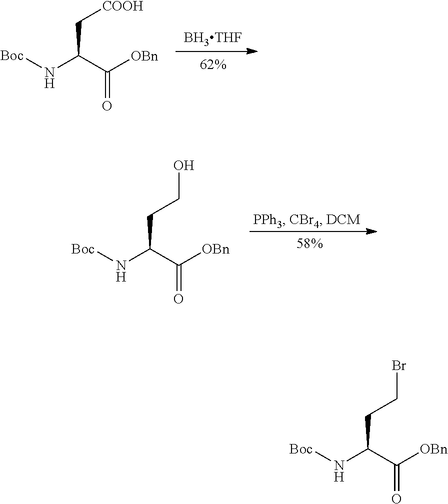

Under K₂CO₃, a nucleophilic substitution of the bromide with benzyloxycarbonyl-protected cytosine and 2-amino-6-chloropurine was performed. In the next step, TFA/H₂O was hydrolysed, with the Boc protective group being removed simultaneously. The subsequent step was carried out hydrogenolytically with PdO-H₂O. This was followed by protection with Boc anhydride into Boc-L-HalG-OH or Boc-D-HalG-OH.

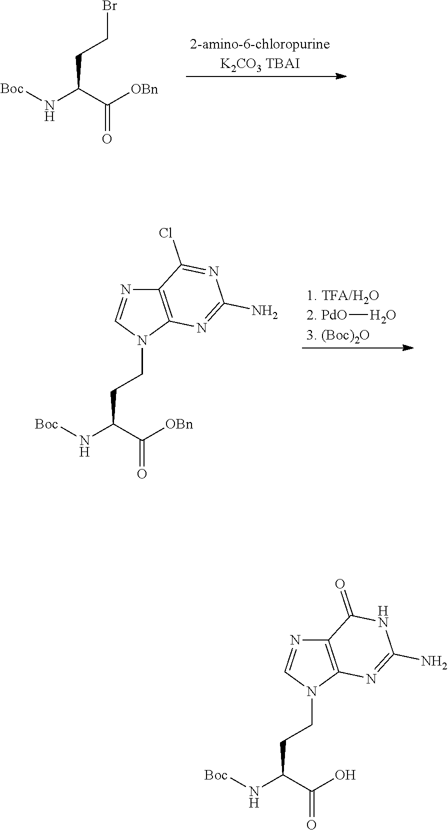

Since the obtaining of the benzyloxycarbonyl protective group of the cytosine base was the desired result, no hydrogenolytic cleavage of the benzyl group could be allowed to occur here. A basic saponification was therefore performed with NaOH/dioxane/H₂O.

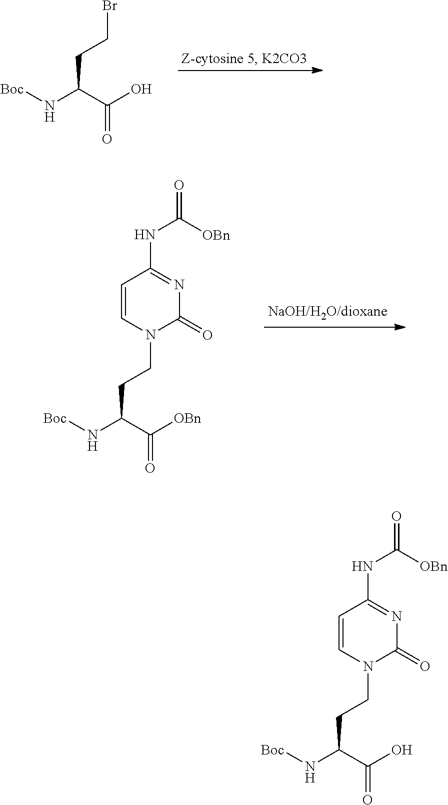 [SMILES: CC(C)(C)OC(=O)N[C@@H](CCBr)C(=O)O ; "Z-cytosine 5, K2CO3" ; CC(C)(C)OC(=O)N[C@@H](CCn1ccc(NC(=O)OCc2ccccc2)nc1=O)C(=O)OCc1ccccc1 ; CC(C)(C)OC(=O)N[C@@H](CCn1ccc(NC(=O)OCc2ccccc2)nc1=O)C(=O)O]

The synthesis of peptide nucleic acids is performed analogously to the synthesis of peptides. The synthesis is performed on solid-state systems. The synthesis can either be carried out using Boc synthesis methods or the Fmoc synthesis method. In this case, the Boc synthesis method was chosen:

HBTU (N-(1-H-benzotriazole-1-yl)-1,1,3,3-tetramethyluronium-hexafluorophosphate) and HOBt (1-hydroxybenzotriazole) were used as coupling reagents for the amino acids. The homoalanyl nucleoamino acids were activated with HATU and HOAt. MBHA-PS resin, overlaid with Boc-L-Lys(2—Cl-Z)-OH, was used as the solid state. Depending on the amino acid, coupling was performed between 35 minutes and two hours. The protective groups of the side chains were selected such that they could be removed simultaneously with the acidic cleaving-off of the resin. For lysine, the side chain was protected with a (2-Cl-Z), for glutaminic acid with a (OBn) and for tyrosine with a (2-Br—Z) group.
-1. Deprotection: 5% m-cresol in TFA (1×5 min, 1×10 min); 2. Washing: DCM/NMP (5×)+pyridine;
-1. Coupling: 5.0 eq. Boc-Hal-OH or 5.0 eq. Boc-AS-OH, 4.5 eq. HATU or HBTU, 5.0 eq. HOAt or HOBt 12 eq. DIPEA, NMP; 2. Washing: DCM/NMP (5×), 10% piperidine in NMP (3×), DCM/NMP (5×);
-1. Capping: Ac20/DIPEA/NMP (1:1:8), (2×5 min); 2. Washing: DCM/NMP (5×), 10% piperidine in NMP (3×), DCM/NMP (5×); Cleavage: TFA/TFMSA/m-cresol (8:1:1)

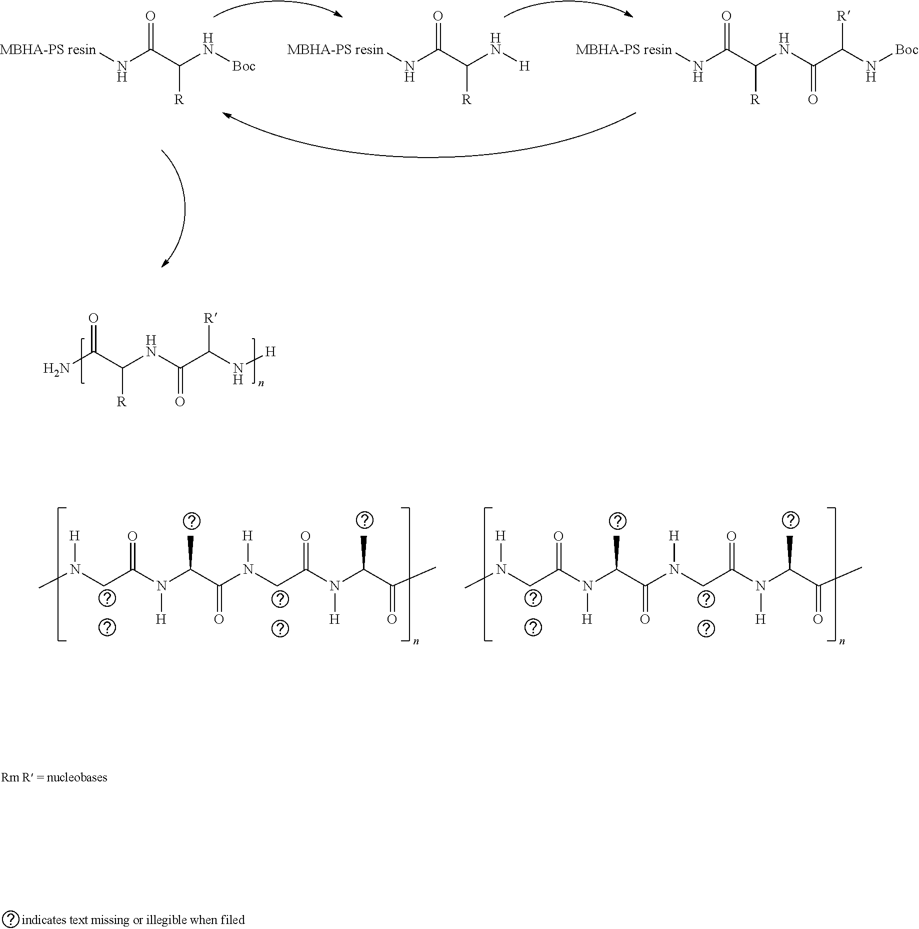

A 15-mer peptide with incorporated nucleoamino acids was prepared using this method.

This was followed by Williamson's ether synthesis by means of K₂CO₃/acetone and a functionalized perfluorocarbon molecule for 48 hours. The perfluorination reached all accessible NH₂ and OH groups, with the reaction occurring analogously to the perfluorination of individual nucleobases and peptide nucleic acid monomers (as described above). By varying the reaction time (2 h to 72 h), the degree of perfluorination was able to be reduced or increased.

### SAMPLE EMBODIMENT 12

### Selection of the Predetermined Breaking Points

The following predetermined breaking points were used here: The fPFC/mRNA complexes are absorbed by endocytosis and packed in lysosomes. During this process, a substantial jump in pH from 7.4 to 7.2 occurs in the extracellular space to up to 4.0 in the lysosome that is caused by an ATP-dependent proton pump (Serresi et al. 2009). This low pH value of 4.0 is crucial for the selection of the predetermined breaking point. There is a series of acid-labile predetermined breaking points that are worthy of consideration for the fPFC system (Warnecke, 2008, Warnecke 2010). However, glycosidic bonds at the 2′ position hydrolyse at low pH values; see diagram 14.

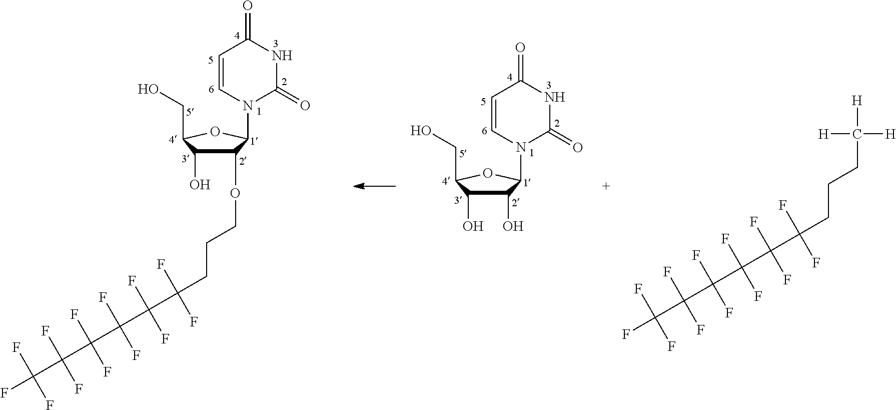

### Diagram 14

Since perfluorohydrocarbons mask the molecule from hydrolases, chemical hydrolysis occurs, so that the linking of harmful OH groups to the perfluorohydrocarbon chain is prevented. The cleaved-off perfluorohydrocarbon chain thus remains inert and does not combine with cell molecules. However, predetermined breaking points that break in the cytoplasm are also useful (approx. pH values=7).

### SAMPLE EMBODIMENT 13

### Alternative Synthesis of Perfluorinated Oligonucleotides and Nucleic Acids

The perfluorination of entire oligimers and nucleic acids is also possible, for example using acid-catalytic methods with fluoric acid or the polymerization of perfluorinated nucleotides using polymerases (Polymerase Chain Reaction).

### SAMPLE EMBODIMENT 14

### Preparation of an Emulsion

A) To set certain particle sizes, it may be necessary to use surfactants. Pluronic F-68 was used here. 5 mg

Pluronic F-68 is dissolved in 10 ml distilled water. 0.5 ml of a functionalized perfluorocarbon/mRNA solution (1.0 g/1.0 microliter) was added to this. Sonification is then performed for 3 cycles, intensity 60. The obtained emulsion is then centrifuged (1200 RPM/5 min) in order to deposit the excessively large particles. Particles with a particle size of 50-100 nanometers are found in the supernatant above same. These are used.

B) Solvent emulsion: 0.5 ml of a functionalized perfluorocarbon/mRNA solution (1.0 g/1.0 microliter) is added to 2 ml tetrahydrofuran and dissolved therein. This is then brought to 10 ml using distilled water. The emulsion obtained is then centrifuged (1200 RPM/5 min) in order to deposit the excessively large particles. Particles with a particle size of 50-100 nanometers are found in the supernatant above same. These are used.

C) 0.5 ml of a functionalized perfluorocarbon/mRNA solution (1.0 g/1.0 microliter) is added to 10 ml distilled water. Sonification is then performed for 3 cycles, intensity 60. The obtained emulsion is then centrifuged (1200 RPM/5 min) in order to deposit the excessively large particles. Particles with a particle size of 50-100 nanometers are found in the supernatant above same. These are used.

### SAMPLE EMBODIMENT 15

### Preparation of an Artificial mRNA with Therapeutic Sequence for Treating an Acquired Genetic Disease

The artificial perfluorinated mRNA is prepared as described above. The predetermined breaking point of this system is a glycosidic bond. In addition, the GC content of the artificial mRNA is increased while maintaining the same coding information, which increases the life span (resistance to RNAses).

Complexes of artificial mRNA and functionalized perfluorocarbon have both hydrophilic and hydrophobic characteristics and require no additional surfactant; the preparation of the emulsion is done as described under C) of sample embodiment 8. The emulsion worked up in a buffer is processed by an apparatus into an aerosol that is applied as an inhalation spray and gets into the bloodstream of the body via the lung. The complex circulates in the blood and is absorbed non-specifically through endocytosis/pinocytosis. The breakage of the predetermined breaking points occurs in the endosomes and lysosomes of the cell through chemical hydrolysis. The released mRNA and the released perfluorocarbon molecules are released by the endosome or by the lysosome into the cytoplasm. The translation of the mRNA and the formation of the therapeutic proteins occur in the cytoplasm. The transport system (perfluorocarbon molecule) is inert and cannot react with cell molecules. Due to its vapor pressure and other physical properties, the transport system is excreted via the lung and kidney function.

### SAMPLE EMBODIMENT 16

### Release of Therapeutic siRNA in Cancer Treatment

The preparation of the siRNA and the linking of transferrin to the system is done as described above, as is the preparation of the emulsion. This emulsion is administered intravenously. By virtue of the ligand transferrin, the particles are absorbed especially strongly in tumor cells, which enables treatment with siRNA in specific target cells. The predetermined breaking points are broken in the endosomes or the lysosomes through chemical hydrolysis. siRNA and perfluorocarbon molecules are released into the cytoplasm. The perfluorocarbon molecules are excreted via the lung and kidney function.

### SAMPLE EMBODIMENT 17

### A Therapeutic Vaccination Against HPV Type 16

siRNA for shutting down a specific gene expression of HVP type 16 and emulsion thereof is prepared as described above. This emulsion is processed in a buffer into a tincture that is applied to the mucosa. The complexes penetrate into the tissue and are absorbed by the cells through endocytosis/pinocytosis. The path of action of the siRNA and path of elimination of the perfluorocarbon molecules are as described above.

### SAMPLE EMBODIMENT 18

### Detection of the Absorption of Perfluorinated Nucleic Acid into the Cell

To follow the path of the perfluorinated nucleic acids into the cell, the following construct with rhodamine linking was used:

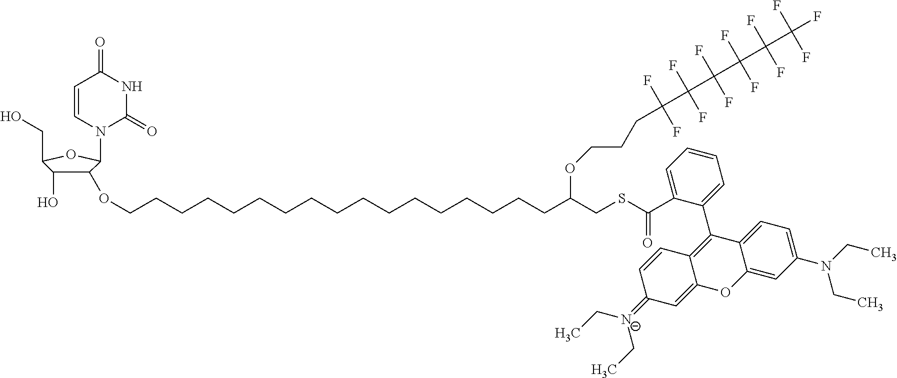

The compound was first dissolved in tetrahydrofuran (THF), and this solution was subsequently titrated in isopropanol. The obtained particles had an average size of 50 nm.

Cells of line HEK 293 were used as cell culture Transfection was performed one day after cell enlargement. As a control, a transfection was performed with pure rhodamine particles of equivalent rhodamine concentration to test the perfluorinated nucleic acids. Immediately after transfection, the cell culture medium of the cells with perfluorinated nucleic acids appeared clear and unchanged. The medium with rhodamine appeared slightly cloudy and reddish. One explanation of this is possible that the rhodamine is bound to the PFC particle in the perfluorinated nucleic acids and sinks to the bottom as a small particle, whereas the dye is completely dissolved in the medium with pure rhodamine.

20 minutes after transfection, a homogeneous coloration could be observed over the cell surface in the cells with perfluorinated nucleic acids. In the cells with pure rhodamine, no defined coloration could be seen at this point in time.

24 hours after transfection, the coloration of the cells with perfluorinated nucleic acids had changed compared to the 20 minute mark: The uniformly homogeneous coloration on the cell surface could no longer be observed. Instead, a granular coloration was observed whose vesicles had elevated color intensity, whereas the intermediate spaces hardly exhibited any coloration.

During the investigation of this process using images on the confocal microscope (see FIG. 1), it became evident that the perfluorinated nucleic acids were transported in the endosomes and lysosomes. The endosomes and lysosomes filled with perfluorinated nucleic acids were detected throughout the cell cytoplasm. The particles could not be found in the nucleus (nucleus localization sequence or cell division required). In addition, it was observed that a quantity of particles released from the endosornes/lysosomes was already located in the cytoplasm, which was apparent in the diffuse coloration outside of the vesicle.

In contrast, in the transfection with pure rhodamine, while a coloration was also observed after 24 hours, it was substantially weaker and diffuse in comparison on the cell surface instead of in the cytoplasm and granular.

The transfected cells were also studied using FACS (Fluorescence Activated Cell Sorting) (see FIG. 2). The FACS studies were conducted on cells transfected with pure rhodamine (control) and with perfluorinated nucleic acids, and the results of confocal microscopy were confirmed. Perfluorinated nucleic acids are indeed absorbed by the cell, packed in endosomes and lysosomes, and released by these into the cytoplasm.

As a result of the high level of effectiveness of the absorption of perfluorinated nucleic acids into the cell, this method constitutes a true alternative to virally mediated gene transfer and to other methods for transferring nucleic acids and analogs thereof (modified nucleic acids, peptide nucleic acids) into the cell.

1. Compound of the general formula (I)
A-B-C(F′,G′)-D-E-F-G-A′ (I)
or of the general formula (II)
A-B-C(F′,G′)-D-B-E-F-G-A′ (II)
wherein
-A is at least one molecule selected from the group consisting of perfluorocarbons (PFCs), perfluorosilicon compounds and other perfluorinated compounds,
-B is at least one predetermined breaking point in the form of a physically, chemically or enzymatically several bond,
-C is absent or at least one linker molecule,
-D is absent or at least one spacer molecule,
-E is at least one molecule selected from the group consisting of nucleobases, nucleosides, nucleotides, oligonucleotides, nucleic acids, modified nucleobases, modified nucleosides, modified nucleotides, modified oligonucleotides, modified nucleic acids, peptide nucleic acid monomers, peptide nucleic acid oligomers, peptide nucleic acids and other nucleic acid analogs,
-F, F′ is absent or at least one ligand or a recognition sequence,
-G, G′ is absent or at least one marker molecule,
-A′ is absent or has the meaning of A, and wherein the compounds 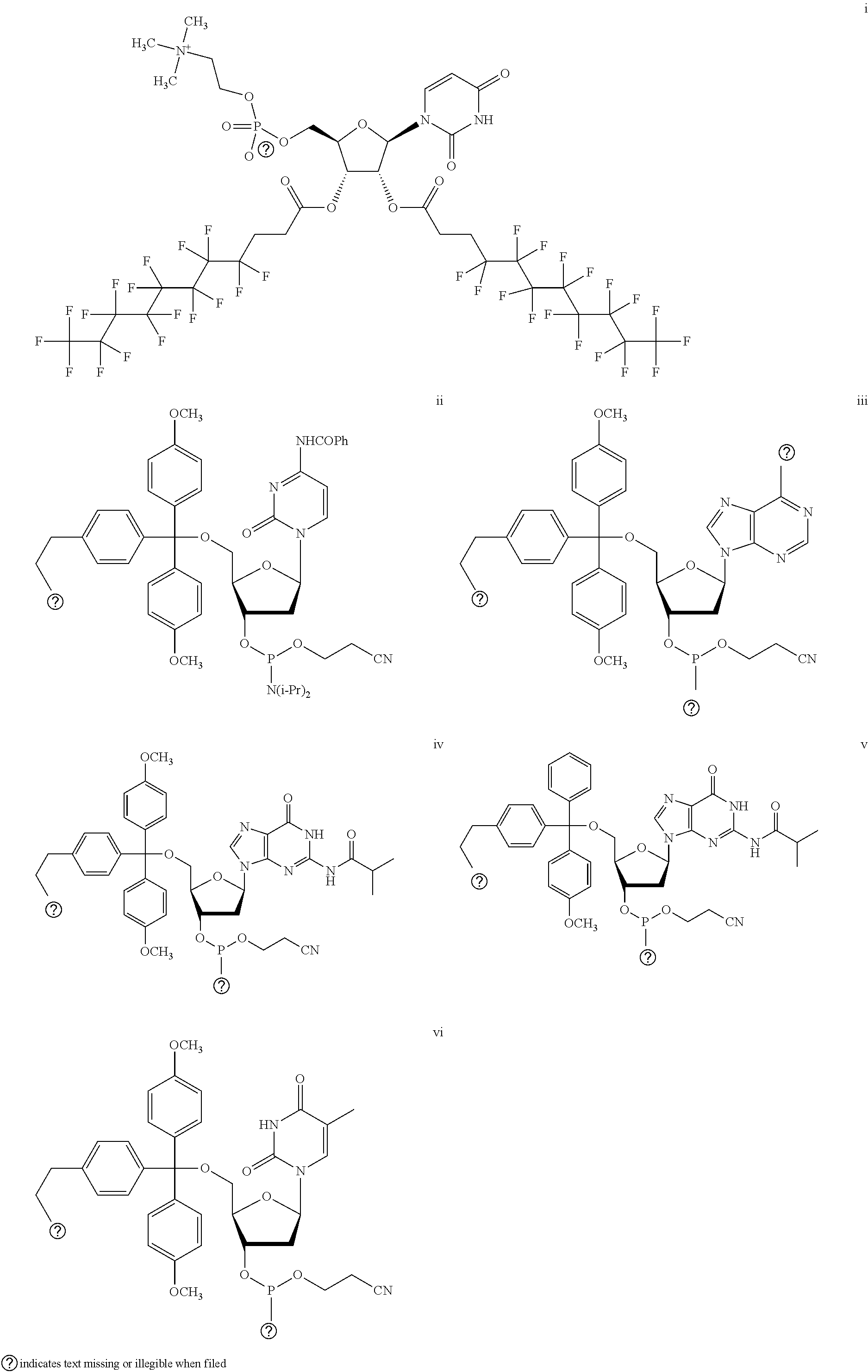
are excluded.

2. Compound as set forth in claim 1, wherein A is at least one molecule selected from the group consisting of perfluorocarbons (PFCs) containing straight or branched acyclic or cyclic, polycyclic or heterocyclic aliphatic alkanes, alkenes, alkines, aromatic compounds and combinations of these compounds in which all of the H-atoms are substituted by F-atoms, which optionally also contain at least one non-fluorinated or partially fluorinated substituent in the form of one or more functional groups or heteroatoms, or these in conjunction with one or more additional functional groups.

3. Compound as set forth in claim 2, wherein A is selected from the group consisting of perfluorocarbons (PFCs) containing C₁-C₂₀ alkanes, alkenes or alkines which can be linear, branched, cyclic, polycyclic or heterocyclic; and perfluorocarbons (PFCs) containing C₆-C₅₀ aromatic or heteroaromatic systems.

4. Compound as set forth in claim 1, wherein A is at least one molecule selected from the group consisting of perfluorocarbons (PFCs) containing straight or branched acyclic or cyclic, polycyclic and heterocyclic aliphatic silanes, in which all of the H-atoms are substituted by F-atoms which optionally also contain non-fluorinated or partially fluorinated substituents with one or more functional groups or heteroatoms or these in conjunction with one or more additional functional groups.

5. Compound as set forth in claim 1, wherein A contains two or more molecules selected from the group consisting of perfluorocarbons (PFCs), perfluorosilicon compounds and other perfluorinated compounds.

6. Compound as set forth in claim 1, wherein the at least one predetermined breaking point B is embodied in the form of an acid-labile group a plasmalogen perfluoride, a vinyl ether group or orthoester.

7. Compound as set forth in claim 1, wherein at least one linker molecule C is selected from the group consisting of straight or branched acyclic or cyclic, polycyclic or heterocyclic aliphatic alkanes, alkenes, alkines, aromatic compounds and combinations of these compounds with functional groups.

8. Compound as set forth in claim 1, wherein at least one spacer molecule D is selected from the group consisting of straight and branched aliphates with one or more functional groups, optionally having spacer molecule D as linker molecule C.

9. Compound as set forth in claim 1, wherein the molecule E is selected from the group of nucleobases consisting of adenine, guanine, hypoxanthine, xanthine, cytosine, uracil, thymine, and a modified nucleobase.

10. Compound as set forth in claim 1, wherein molecule E is selected from the group of the nucleosides consisting of adenosine, guanosine, cytidine, 5-methyluridine, uridine, deoxyadenosine, deoxyguanosine, thymidine, deoxyuridine, deoxycytidine and a modified nucleoside.

11. Compound as set forth in claim 1, wherein molecule E is selected from the group of the nucleotides consisting of AMP, GMP, m5UMP, UMP, CMP, dAMP, dGMP, dTMP, dUMP, dCMP, cAMP, cGMP, c-di-GMP, cADPR, ADP, GDP, m5UDP, UDP, CDP, dADP, dGDP, dTDP, dUDP, dCTP, ATP, GTP, m5UTP, UTP, CTP, dATP, dGTP, dTTP, dUTP, dCTP, modified nucleotides that originate from the above-described building blocks, nucleotides with modifications on the sugar-phosphate structure, zwitterionic oligonucleotides and nucleotides in which the phosphate has been replaced by methyl phosphonate or a dimethyl sulfone group.

12. Compound as set forth in claim 1, wherein the molecule E is selected from the group consisting of single-stranded and double-stranded oligonucleotides and nucleic acids, these having a length of from 2 base pairs to greater than 1,000,000 bp.

13. Compound as set forth in claim 1, wherein the at least one ligand F, F′ is selected from the group consisting of transferrin, folic acid, galactose, lactose, mannose, epidermal growth factor, RGD peptides, biotin, and other substances that enable a specific entry of the present compound into the cell or a nucleus localization sequence.

14. Compound as set forth in claim 1, wherein the at least one marker molecule G, G′ is selected from the group consisting of fluorescent dyes, peroxidase dyes and other substances that enable the molecule to be followed in metabolism.

15. A method of non-viral transfer of at least one molecule E into at least one cell of a eukaryotic organism comprising administering a compound set forth in claim 1 to said eukaryotic organism.

16. Pharmaceutical composition comprising at least one compound as set forth in claim 1 and at least one surface-active substance.

17. Pharmaceutical composition as set forth in claim 16, wherein the composition is present in the form of a dispersion, suspension, emulsion or solution with an average particle size between 2 nm and 200 μm.

18. A method of non-viral transfer of at least one molecule E into at least one cell of a eukaryotic organism comprising administering a compound set forth in claim 1 to said eukaryotic organism.

19. A Compound of claim 2, wherein the heteroatom is selected from the group consisting of Br, I, Cl, H, Si, N, O, S and P.

20. A compound of claim 6, wherein the acid-labile group is selected from the group consisting of a glycosidic bond, a disulfide bridge, an ester group, ether group, peptide bond, imine bond, hydrazone bond, acylhydrazone bond, ketal bond, acetal bond, cis-aconitrile bond, trityl bond, beta-D-glucosylceramide, and dithiothreitol.
